SEQUENCE LISTING (6 sequences)

SEQ ID NO: 1 - protein, 68 aa, Artificial Sequence
  Synthetic Polypeptide
KPVSLLYRCPCRFFESHVARANVKHLKILNTPNCALQIVARLKNNNRQVCIDPKLKWIQEYLEKALNK

SEQ ID NO: 2 - protein, 89 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 3 - protein, 89 aa, Rattus norvegicus
MDAKVVAVLALVLAALCISDGKPVSLSYRCPCRFFESHVARANVKHLKILNTPNCALQIVARLKSNNRQVCIDPKLKWIQEYLDKALNK

SEQ ID NO: 4 - DNA, 1940 bp, Homo sapiens
gccgcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagtaagcacaacagccaaaaaggactttccgctagacccactcgaggaaaactaaaaccttgtgagagatgaaagggcaaagacgtgggggagggggccttaaccatgaggaccaggtgtgtgtgtggggtgggcacattgatctgggatcgggcctgaggtttgccagcatttagaccctgcatttatagcatacggtatgatattgcagcttatattcatccatgccctgtacctgtgcacgttggaacttttattactggggtttttctaagaaagaaattgtattatcaacagcattttcaagcagttagttccttcatgatcatcacaatcatcatcattctcattctcattttttaaatcaacgagtacttcaagatctgaatttggcttgtttggagcatctcctctgctcccctggggagtctgggcacagtcaggtggtggcttaacagggagctggaaaaagtgtcctttcttcagacactgaggctcccgcagcagcgcccctcccaagaggaaggcctctgtggcactcagataccgactggggctgggcgccgccactgccttcacctcctctttcaacctcagtgattggctctgtgggctccatgtagaagccactattactgggactgtgctcagagacccctctcccagctattcctactctctccccgactccgagagcatgcttaatcttgcttctgcttctcatttctgtagcctgatcagcgccgcaccagccgggaagagggtgattgctggggctcgtgccctgcatccctctcctcccagggcctgccccacagctcgggccctctgtgagatccgtctttggcctcctccagaatggagctggccctctcctggggatgtgtaatggtccccctgcttacccgcaaaagacaagtctttacagaatcaaatgcaattttaaatctgagagctcgctttgagtgactgggttttgtgattgcctctgaagcctatgtatgccatggaggcactaacaaactctgaggtttccgaaatcagaagcgaaaaaatcagtgaataaaccatcatcttgccactaccccctcctgaagccacagcagggtttcaggttccaatcagaactgttggcaaggtgacatttccatgcataaatgcgatccacagaaggtcctggtggtatttgtaactttttgcaaggcatttttttatatatatttttgtgcacatttttttttacgtttctttagaaaacaaatgtatttcaaaatatatttatagtcgaacaattcatatatttgaagtggagccatatgaatgtcagtagtttatacttctctattatctcaaactactggcaatttgtaaagaaatatatatgatatataaatgtgattgcagcttttcaatgttagccacagtgtattttttcacttgtactaaaattgtatcaaatgtgacattatatgcactagcaataaaatgctaattgtttcatggtataaacgtcctactgtatgtgggaatttatttacctgaaataaaattcattagttgttagtgatggagcttaaaaaaaa

SEQ ID NO: 5 - DNA, 292 bp, Rattus norvegicus
catggacgccaaggtcgtcgctgtgctggccctggtgctggccgcgctctgcatcagtgacggtaagccagtcagcctgagctacagatgcccctgccgattctttgagagccatgtcgccagagccaacgtcaaacatctgaaaatcctcaacactccaaactgtgcccttcagattgttgcaaggctgaaaagcaacaacagacaagtgtgcattgacccgaaattaaagtggatccaagagtacctggacaaagccttaaacaagtaagcacaacagcccaaaggactt

SEQ ID NO: 6 - DNA, 3948 bp, Artificial Sequence
  Synthetic Polynucleotide
agatctcctagggagtccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgccatcggtgaccactagtggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatcggtaccaagcttgccaccaccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacaccccaaactgtgcccttcagattgtagcccggctgaagaacaacaacagacaagtgtgcattgacccgaagctaaagtggattcaggagtacctggagaaagccttaaacaagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagggccgcggtggccatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatc

(19) United States
(12) Patent Application Publication
(10) Pub. No.: US 2018/0296643 A1
(43) Pub. Date: Oct. 18, 2018

(54) SDF-1 FOR ANAL AND SPHINCTER WOUND HEALING

(71) Applicant: The Cleveland Clinic Foundation, Cleveland, OH (US)

(72) Inventor: Massarat Zutshi, Cleveland, OH (US)

(21) Appl. No.: 15/955,221

(22) Filed: Apr. 17, 2018

Related U.S. Application Data
(60) Provisional application No. 62/486,038, filed on Apr. 17, 2017.

(51) Int. Cl. A61K 38/19; A61K 9/00; C12N 15/70; C07K 14/52; A61K 48/00; C12N 15/79; A61P 43/00

(52) U.S. Cl. CPC A61K 38/195; A61K 9/0024; C12N 15/70; A61K 9/0014; A61K 2300/00; A61K 48/0075; A61K 48/005; C12N 15/79; A61P 43/00; C07K 14/522

(57) ABSTRACT

A method for treating an anal or sphincter wound of a subject is described. The method includes administering a therapeutically effective amount of a stromal cell-derived factor-1 (SDF-1) protein or protein variant, or an SDF-1 or SDF-1 variant expression vector in or proximate to the anal or sphincter wound. Topical formulations for administering the SDF-1 or SDF-1 expression vector to an anal or sphincter wound are also described.

### CONTINUING APPLICATION DATA

This application claims the benefit of U.S. Provisional Application Ser. No. 62/486,038, filed Apr. 17, 2017, the disclosure of which is incorporated by reference herein.

### GOVERNMENT FUNDING

This invention was made with government support under W81XWH-13-2-0052 awarded by the Department of Defense. The government has certain rights in this invention.

### SEQUENCE LISTING

The instant application contains a Sequence Listing which has been submitted electronically in ASCII format and is hereby incorporated by reference in its entirety. Said ASCII copy, created on Apr. 13, 2018, is named SDF-1_ST25 and is 10,975 bytes in size.

### BACKGROUND

Factors that are responsible for fecal leakage include a disrupted external and/or internal anal sphincter and changes in bowel motility, rectal capacity, and sensation. Current cell-based animal research has focused on repairing a defect of the anal sphincters. Salcedo et al. Stem Cell Res., 10:95-102 (2013). Although none of the studies have evaluated muscle morphology, most studies have reported filling of small to-moderate defects with muscle. Lorenzi et al. Dis Colon Rectum, 51:411-420 (2008). Some studies evaluated muscle tensile strength in the regenerated muscle. Pathi et al., Obstet Gynecol., 119:134-144 (2012) All of these studies, however, have reported findings in the setting of an acute injury. However, fecal incontinence most often manifests many years after an injury, which may have occurred after childbirth, surgical trauma, radiation, or neurologic etiologies, among others, and hence there is a need to research regeneration of deficient muscle long after the injury.

Cell-based therapies for fecal incontinence have been explored in the last decade, which are attributable to research advances that have suggested the regenerative potential of stem cells. Fu et al., Urology, 75, 718-723 (2010) Current treatment requires a multimodal approach that focuses on medical and surgical management. Although medical management involves diet/bowel management and biofeedback measures, surgical managements are based on bulking (injectables and radiofrequency treatments), changing motility via neuromodulation (sacral nerve or percutaneous tibial nerve stimulation), anatomic repair of the anal sphincter (sphincteroplasty), and augmenting the anal sphincter (artificial anal sphincter and magnetic anal sphincter). During an acute injury, signaling cytokines expressed at the site of injury cause chemoattraction of stem cells, thereby initiating the process of repair. Barussaud et al., Colorectal Dis., 15:1499-1503 (2013); Kucia et al., J Mol Histol., 35:233-245 (2004). At the site of an injury that has occurred in the remote past, as in fecal incontinence, these signals no longer exist, and there is a need for re-expression to achieve a successful regenerative outcome. There are a few available means to re-express the cytokine signal to mimic the milieu of an acute injury. Shinohara et al., J Orthop Res., 29:1064-1069 (2011). One is to exogenously introduce the cytokines at the site of the intended repair (Sundararaman et al., Gene Ther., 18:867-873 (2011)), and the other is to inflict a conditioned injury to create a microenvironment that mimics that of an acute injury. Sun et al., Dis Colon Rectum., 59:434-442 (2016).

### SUMMARY OF THE INVENTION

The inventors have demonstrated that an SDF-1 expression vector can be used to stimulate regeneration of the anal sphincter in both rat and pig animal models. Accordingly, in one aspect, the invention provides a method for treating an anal or sphincter wound of a subject. The method includes administering a therapeutically effective amount of a stromal cell-derived factor-1 (SDF-1) protein or protein variant, or an SDF-1 or SDF-1 variant expression vector in or proximate to the anal wound. In some embodiments, the anal wound is a chronic anal wound, while in further embodiments the anal wound is an anal sphincter wound. In yet further embodiments, the anal wound is a muscle defect. In some embodiments, the SDF-1 protein or SDF-1 expression vector is injected into the wound or an area proximate to the wound.

The SDF-1 or SDF-1 variant can be administered as either a protein or a polynucleotide capable of expressing SDF-1 or an SDF-1 variant. In some embodiments, an SDF-1 expression vector is administered to the subject. For example, the SDF-1 expression vector can be a plasmid vector, such as the SDF-1 expression vector comprising SEQ ID NO: 6. In other embodiments, the SDF-1 protein is administered to the subject. For example, an SDF-1 protein comprising SEQ ID NO: 1 can be administered to the subject.

The SDF-1 protein or SDF-1 expression vector can be administered alone, or can be administered with other cells or compounds useful for wound healing. In some embodiments, the method further comprises administering mesenchymal stem cells in or proximate to the anal wound. In further embodiments, the SDF-1 protein or SDF-1 expression vector is administered as a topical formulation. For example, in some embodiments, the topical formulation comprises a hydrogel scaffold.

Another aspect of the invention provides a topical formulation for treating an anal or sphincter wound. The topical formulation comprises a topical pharmaceutical carrier and an SDF-1 protein or protein variant, or an SDF-1 or SDF-1 variant expression vector. In some embodiments, the SDF-1 included in the formulation is a protein, while in other embodiments the SDF-1 is included as an expression vector. For example, the SDF-1 protein comprising SEQ ID NO: 1 can be included, or the expression vector can be a plasmid vector, such as the SDF-1 expression vector comprising SEQ ID NO: 6. In some embodiments, the topical formulation further comprises mesenchymal stem cells, while in other embodiments, the topical pharmaceutical carrier comprises a hydrogel scaffold.

### BRIEF DESCRIPTION OF THE FIGURES

The present invention may be more readily understood by reference to the following figures, wherein:

FIG. 1 provides images illustrating the experimental design of a rat model of a chronic large anal sphincter defect undergoing various interventions. The anal sphincter injury is shown as a pink dual arrow; a sample of the anal manometry resting pressure is shown on the bottom left, and a histological sample stained with Masson trichrome for gross and structural analysis is shown on the bottom right. Scale bar=500 μm. EAS=external anal sphincter; IAS=internal anal sphincter.

FIGS. 2A-2B provide graphs and images providing A, Representative in vivo serial bioluminescence images of the left lower back in a rat model of gluteus muscle injury, in groups receiving luciferase-encoded plasmid in a dose of 100 μg (LUC100) or 200 μg (LUC200) or phosphate-buffered saline solution (PBS) injected into the wound wall soon after injury (IVIS Lumina II at field-of-view A), and B, The line plot shows the significantly higher signal of both LUC100 and LUC200 vs PBS (*p<0.05), as well as a significantly higher signal of LUC200 vs LUC100 (#p<0.05) using 2-way ANOVA followed by the Tukey test. Each scatter plot on the line plot represents the mean±SEM from 6 individual animals. Radiance p/sec/cm²/st=photons per second per centimeter squared per radiation.

FIG. 3 provides a graph showing anal resting pressures at individual time points (pre-excision (Pre-exc), pretreatment (Pre-tx), and 4 weeks post-treatment (Post-tx)) for the treatment groups, with lines connecting measurements from within the same animal. The graph indicates that, at the post-excision time point, both pSDF-1 and pSDF-1+S&MSC groups achieved a significant improvement in anal pressure than at the pretreatment time point (2-way ANOVA followed by Turkey test, *p<0.05). IA=injury without treatment; pSDF-1=100 μg of stromal derived factor 1-encoded plasmid injected at the site of the defect; pSDF-1+MSC=both pSDF-1 and 800,000 of MSCs injected at the site of the defect; pSDF-1+S&MSC=pSDF-1 injected at the site of the defect with injection of a gelatin scaffold mixed with MSCs; MSC=mesenchymal stem cell.

FIG. 4 provides representative pictures of transverse anal sphincter sections stained by Masson trichrome and 4 weeks after various treatments after a partial anal sphincter excision treated 3 weeks after injury. In the area of the defect (circled), muscle is indicated by a red arrow and connective tissue is indicated by a yellow arrow. A higher percentage of muscle is seen at the area of injury compared with the uninjured normal muscle in the same section noted in all 3 groups with treatment (pSDF-1, pSDF-1+MSC, pSDF-1+S&MSC) vs the IA group. IA=injury without treatment; pSDF-1=stromal derived factor 1-encoded plasmid local injection at the site of the defect; pSDF-1+MSC=pSDF-1 and MSC injected at the site of the defect; pSDF-1+S&MSC=pSDF-1 injected at the site of the defect with insertion of a gelatin scaffold mixed with MSC; MSC=mesenchymal stem cell. Scale bar=500 pa.

FIGS. 5A-5B provide graphs showing A, Results of quantification of new muscle in the area of injury normalized to muscle in the uninjured area in the same animal after different treatment. Each bar represents the percentage as mean±SEM from 8 animals receiving the plasmid (pSDF-1, pSDF-1+MSC, and pSDF-1+S&MSC) that had a significantly higher ratio of muscle at the area of injury equated with the normal half in the same section compared with the injury-alone (IA) group (1-way ANOVA followed by Tukey test, *p<0.05). B, Results of quantification of new connective tissue in the area of injury normalized to connective tissue in the uninjured area in the same animal after various treatments. Each bar represents the percentage as mean±SEM from 8 individual animals. There was no significant difference among groups in the connective tissue in the area of injury 4 weeks after each treatment (1-way ANOVA followed by Tukey test, *p<0.05). IA=injury without treatment; pSDF-1=stromal derived factor 1-encoded plasmid local injection at the site of the defect; pSDF-1+MSC=pSDF-1 and MSC injected at the site of the defect; pSDF-1+S&MSC=pSDF-1 injected at the site of the defect with insertion of a gelatin scaffold mixed with MSC; MSC=mesenchymal stem cell.

FIG. 6 provides an image showing representative immunohistochemistry staining using anti-Desmin of a rat anal canal section. Smooth muscle is stained light brown (green arrow), and striated muscle is stained dark brown (red arrow). Scale bar=50 μm.

FIG. 7 provides a bar chart showing the resting anal pressure (cm H₂O), at preinjury, as well as pretreatment and posttreatment time points among groups. Each bar represents the mean±SD from 8 individual animals. *Eight weeks after treatment, all 3 treatment groups had significantly higher anal resting pressure than the IA group. IA=injury without treatment; P=plasmid injected at the site of the defect; P+MSC=plasmid and mesenchymal stem cells (MSC) injected at the site of the defect; P+S&MSC=plasmid injected at the site of the defect with a gelatin scaffold mixed with MSCs.

FIG. 8 provides an image showing a representative cross-sections of rat anal canals 8 weeks after different treatments after a chronic injury. The top row shows sections stained by Masson trichrome. The section shows the outermost layer as a white serosa; the next layer is a lighter pink and is the external anal sphincter muscle. The inner muscle layer stained a darker pink is the internal anal sphincter muscle, and the submucosa is stained blue. The mucosal lining is stained blue. In the area of the defect (indicated by red dotted lines), muscle is indicated by an orange arrow; connective tissue is indicated by a green arrow. More muscle is seen at the area of injury in all 3 groups receiving the stromal cell-derived factor 1 plasmid treatment compared with IA group. The bottom row shows Desmin staining of all groups with both skeletal and smooth muscles stained brown. Skeletal muscle is stained darker, whereas smooth muscle has a lighter stain. Scale bar=1 mm IA=injury without treatment; P=plasmid injected at the site of the defect; P+MSC=plasmid and mesenchymal stem cells (MSC) injected at the site of the defect; P+S&MSC=plasmid injected at the site of the defect with a gelatin scaffold mixed with MSCs.

FIGS. 9A-9B provide graphs showing A, Proportion of muscle in the area of injury normalized to the uninjured area in the same animal 8 weeks after treatment. *Group receiving the plasmid alone (P) had significantly higher muscle in the area of injury normalized to the intact muscle in the same section compared with the injury alone (IA) group. B, Proportion of connective tissue in the area of injury normalized to the uninjured area in the same section 8 weeks after treatment. #Group receiving the plasmid alone (P) had significantly lower connective tissue in the area of injury normalized to the intact tissue in the same section compared with the IA group and the group receiving plasmid injected at the site of the defect with a gelatin scaffold mixed with mesenchymal stem cells (MSC; P+S&MSC). Each bar represents mean±SD of data from 8 animals. P+MSC=plasmid and MSC injected at the site of the defect.

FIGS. 10A-10B provide graphs showing the quantification of skeletal muscle of the external anal sphincter (A) and smooth muscle of the internal anal sphincter (B) in the area of injury normalized to the uninjured area in the same animal 8 weeks after treatment in each group. No significant differences were observed between the groups. Each bar represents mean±SD of data from 8 animals. IA=injury without treatment; P=plasmid injected at the site of the defect; P+MSC=plasmid and mesenchymal stem cells (MSC) injected at the site of the defect; P+S&MSC=plasmid injected at the site of the defect with a gelatin scaffold mixed with MSC.

FIGS. 11A-11B provide graphs showing the Western blot results of CXCR4 (A) and MyF5 (B) expression normalized to the mean of the injury alone (IA) group 7 days after treatment. No significant differences between the groups was noted. Each bar represents mean±SD of data from 6 animals. P=plasmid injected at the site of the defect; P+MSC=plasmid and mesenchymal stem cells (MSC) injected at the site of the defect; P+S&MSC=plasmid injected at the site of the defect with a gelatin scaffold mixed with MSC.

FIG. 12 provides a schematic image showing the ACL-01110Sk plasmid for expressing SDF-1.

FIG. 13 provides a bar chart and the data table showing the posterior resting anal pressure (cm H₂O) at pre-injury, as well as pre-treatment and post-treatment time points among groups. Each bar represents the mean±SD from individual animal groups and the One-Way ANOVA followed Tukey test was used. *After treatment, both 1-SDF-1(p=0.003) and 2-SDF-1 (p=0.004) groups had significantly higher pressure compared to saline group; #1-SDF-1 group had significantly higher pressure at post-treatment than pre-treatment time point (p<0.001); Saline: local saline injection at 6-week post defect surgery. 1-SDF-1: local SDF-1 injection at 6-week post defect surgery. 2-SDF-1: local SDF-1 injection at 6-week and 8-week post defect surgery.

FIG. 14 provides a bar chart showing the average resting anal pressure (cm H₂O) at pre-injury, as well as pre-treatment and post-treatment time points among groups. Each bar represents the mean±SD from individual animal groups and the One-Way ANOVA followed Tukey test was used. At post-treatment, #1-SDF-1 group had significantly higher pressure than pre-treatment time point (p<0.001). Saline: local saline injection at 6-week post defect surgery. 1-SDF-1: local SDF-1 injection at 6-week post defect surgery. 2-SDF-1: local SDF-1 injection at 6-week and 8-week post defect surgery.

### DETAILED DESCRIPTION OF THE INVENTION

Unless otherwise defined, all technical and scientific terms used herein have the same meaning as commonly understood by one of ordinary skill in the art to which these exemplary embodiments belong. The terminology used in the description herein is for describing particular exemplary embodiments only and is not intended to be limiting of the exemplary embodiments. As used in the specification and the appended claims, the singular forms “a,” “an,” and “the” are intended to include the plural forms as well, unless the context clearly indicates otherwise. All publications, patent applications, patents, and other references mentioned herein are incorporated by reference in their entirety.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

“Treating”, as used herein, means ameliorating the effects of, or delaying, halting or reversing the progress of a wound or injury. The word encompasses reducing the severity of a symptom of a wound or injury and/or the frequency of a symptom of the wound or injury. A subject is successfully “treated” for a wound or injury if the subject shows observable and/or measurable reduction in or absence of one or more signs and symptoms of a particular wound or injury.

The language “effective amount” or “therapeutically effective amount” refers to a nontoxic but sufficient amount of the composition used in the practice of the invention that is effective to provide effective treatment in a subject. That result can be reduction and/or alleviation of the signs, symptoms, or causes of a wound or injury, or any other desired alteration of a biological system. An appropriate therapeutic amount in any individual case may be determined by one of ordinary skill in the art using routine experimentation.

A “subject,” as used herein, can be any animal, and may also be referred to as the patient. Preferably the subject is a mammal, such as a research animal (e.g., a monkey, rabbit, mouse or rat) or a domesticated farm animal (e.g., cow, goat, horse, pig) or pet (e.g., dog, cat). In some embodiments, the subject is a human.

The term “therapeutically effective” is intended to qualify the amount of each protein or nucleic acid that will achieve the goal of decreasing disease severity while avoiding adverse side effects such as those typically associated with alternative therapies. As is well known in the medical arts, dosage for any one animal or human depends on many factors, including the subject's size, body surface area, age, the particular composition to be administered, sex, time and route of administration, general health, and other drugs being administered concurrently. Specific dosages of proteins and nucleic acids can be determined readily determined by one skilled in the art. A therapeutically effective amount may be administered in one or more doses.

As used herein, the terms “peptide,” “polypeptide” and “protein” are used interchangeably, and refer to a compound comprised of amino acid residues covalently linked by peptide bonds. A protein or peptide must contain at least four amino acids, unless specified otherwise, and no limitation is placed on the maximum number of amino acids that can comprise the sequence of a protein or peptide. Polypeptides include any peptide or protein comprising four or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “wound healing” refers to a regenerative process with the induction of an exact temporal and spatial healing program comprising wound closure and the processes involved in wound closure. The term “wound healing” encompasses but is not limited to the processes of granulation, neovascularization, fibroblast, endothelial and epithelial cell migration, extracellular matrix deposition, re-epithelialization, and remodeling.

All scientific and technical terms used in the present application have meanings commonly used in the art unless otherwise specified. The definitions provided herein are to facilitate understanding of certain terms used frequently herein and are not meant to limit the scope of the present application.

### Treating an Anal Wound Using SDF-1

In one aspect, the invention provides a method for treating an anal wound of a subject, comprising administering a therapeutically effective amount of a stromal cell-derived factor-1 (SDF-1) protein or protein variant, or an SDF-1 or SDF-1 variant expression vector in or proximate to the anal wound.

An anal wound is a wound that represents an injury to the anus, anal sphincter, or anal canal, which are present in the terminal portion of the lower intestine. As used herein, the term “wound” refers to a disruption of the normal continuity of structures caused by a physical (e.g., mechanical, thermal, electrical) force, or a chemical (e.g., biochemical) means. The term “wound” also encompasses contused wounds, as well as torn, lacerated, open, penetrating, puncture, abrasions, grazes, burns, corrosions, wounds caused by strain, ripping, scratching, pressure, and other types of wounds. Anal wounds include anal injuries, anal trauma, and anal fistula. Two of the more common sources of anal injuries include injuries due to pregnancy (Dudding et al., Ann Surg., 247(2): 224-237 (2008) and injuries resulting from explosive devices used in warfare (Jeganathan et al., Clin Colon Rectal Surg., 31(1), 24-29 (2018).

In some embodiments, the anal wound is a muscle defect. The anal sphincter is primarily muscle, including the superficial region, which includes two flattened planes of muscular tissue, which encircle the anus and meet in front to be inserted into the central tendinous point of the perineum, joining with the superficial transverse perineal muscle and other tissue. A muscle defect can be caused by either acute injury or can manifest a long time after injury, either as a result of earlier trauma or as the result of gradual degradation, such as that which can occur when the subject has a metabolic disorder such as diabetes.

The method of the invention can also be used to treat sphincter wounds. A sphincter is a circular muscle that normally maintains constriction of a natural body passage or orifice and which relaxes as required by normal physiological functioning. The body includes a number of different sphincter muscles, such as the anal sphincter, the esophageal sphincter, the cardiac sphincter, the pyloric sphincter, the ileocecal sphincter, the sphincter of Oddi, the urethral sphincter, and the pupillary sphincter.

The present invention relates to the treatment of an anal wound or sphincter wound in a subject by administering to the wound and/or cells proximate the wound an amount of SDF-1, or SDF-1 variant or SDF-1 expression vector, effective to promote wound healing. Any reference to treatment of “a wound” herein refers to treatment of an anal or sphincter wound.

In accordance with an aspect of the invention, the SDF-1 protein or SDF-1 expression vector can be administered proximate the anal wound to promote wound healing. Localized administration of SDF-1 to tissue facilitates recruitment of stem cells and/or progenitor cells, such as endothelial progenitor cells, expressing CXCR4 and/or CXCR7 to the site of the wound being treated, which can facilitate revascularization of the tissue surrounding and/or proximate the anal wound. In some embodiments, additional agents can be included with the SDF-1, but the inventors have also demonstrated that SDF-1 is independently effective. Accordingly, in some embodiments, the method consists essentially of administering SDF-1 to a subject, where SDF-1 is either administered alone or only with other non-active ingredients such as those found in a pharmaceutical carrier.

In one example, the period of time that the SDF-1 is administered in or proximate to the anal or sphincter wound can be from about onset of the wound and/or tissue injury to about days, weeks, or months after tissue injury. In some embodiments, a plasmid encoding SDF-1 is administered to the anal wound prior to the wound being closed (for example by a suture, glue, or other physical means). Topical and/or local SDF-1 delivery by protein or plasmid is sufficient to increase the rate of healing and anal wound closure. Moreover, the SDF-1 treated wounds tended to have less fibrosis than non-SDF-1 treated wounds, which suggests SDF-1 can mitigate scarring in treated anal wounds

It was also found that immediately after onset of tissue injury, cells in the wound tissue or about the periphery or the border of the wound up-regulate expression of SDF-1. After about 24 hours, SDF-1 expression by the cells is reduced. The SDF-1 can be administered after SDF-1 levels are naturally reduced to regenerate the injury by re-stimulating wound healing. Accordingly, in some embodiments, the SDF-1 protein or protein variant, or SDF-1 or SDF-1 variant expression vector is administered at least one week after the anal injury occurred, while in other embodiments the SDF-1 protein or protein variant, or SDF-1 or SDF-1 variant expression vector is administered at least 30 days after the anal injury occurred. In further embodiments, the SDF-1 protein or protein variant, or SDF-1 or SDF-1 variant expression vector is administered at least 45 days after the anal injury occurred.

### SDF-1 Protein

SDF-1 protein, in accordance with the present invention, can have an amino acid sequence that is substantially similar to a native mammalian SDF-1 amino acid sequence. The amino acid sequence of a number of different mammalian SDF-1 protein are known, including human, mouse, and rat SDF-1 proteins. The human and rat SDF-1 amino acid sequences are about 92% identical. SDF-1 can comprise two isoforms, SDF-1 alpha and SDF-1 beta, both of which are referred to herein as SDF-1 unless identified otherwise.

In some embodiments, an SDF-1 protein comprising SEQ ID NO: 1 is administered to the subject. The SDF-1 can have an amino acid sequence substantially identical to SEQ ID NO: 1. SDF-1 that is expressed as a result of administering an expression vector can also have an amino acid sequence substantially similar to one of the foregoing mammalian SDF-1 proteins. For example, the SDF-1 that is expressed using an expression vector can have an amino acid sequence substantially similar to SEQ ID NO: 2. SEQ ID NO: 2, which substantially comprises SEQ ID NO: 1, is the amino acid sequence for human SDF-1 and is identified by GenBank Accession No. NP954637. The SDF-1 that is expressed can also have an amino acid sequence that is substantially identical to SEQ ID NO: 3. SEQ ID NO: 3 includes the amino acid sequences for rat SDF and is identified by GenBank Accession No. AAF01066.

The SDF-1 protein can also be a variant (i.e., protein variant) of mammalian SDF-1, such as a fragment, analog and derivative of mammalian SDF-1. Such variants include, for example, a polypeptide encoded by a naturally occurring allelic variant of native SDF-1 gene (i.e., a naturally occurring nucleic acid that encodes a naturally occurring mammalian SDF-1 polypeptide), a polypeptide encoded by an alternative splice form of a native SDF-1 gene, a polypeptide encoded by a homolog or ortholog of a native SDF-1 gene, and a polypeptide encoded by a non-naturally occurring variant of a native SDF-1 gene. Reference to SDF-1 protein herein is assumed to include protein variants. SDF-1 protein that does not include variants is referred to herein as native SDF-1.

SDF-1 variants have a peptide sequence that differs from a native SDF-1 polypeptide in one or more amino acids. The peptide sequence of such variants can feature a deletion, addition, or substitution of one or more amino acids of a SDF-1 variant Amino acid insertions are preferably of about 1 to 4 contiguous amino acids, and deletions are preferably of about 1 to 10 contiguous amino acids. Variant SDF-1 polypeptides substantially maintain a native SDF-1 functional activity. Examples of SDF-1 polypeptide variants can be made by expressing nucleic acid molecules within the invention that feature silent or conservative changes. One example of an SDF-1 variant is listed in U.S. Pat. No. 7,405,195, which is herein incorporated by reference in its entirety.

SDF-1 polypeptide fragments corresponding to one or more particular motifs and/or domains or to arbitrary sizes, are within the scope of the present invention. Isolated peptidyl portions of SDF-1 can be obtained by screening peptides recombinantly produced from the corresponding fragment of the nucleic acid encoding such peptides. For example, an SDF-1 polypeptides may be arbitrarily divided into fragments of desired length with no overlap of the fragments, or preferably divided into overlapping fragments of a desired length. The fragments can be produced recombinantly and tested to identify those peptidyl fragments that can function as agonists of native CXCR-4 polypeptides.

Variants of SDF-1 polypeptides can also include recombinant forms of the SDF-1 polypeptides. Recombinant polypeptides preferred by the present invention, in addition to SDF-1 polypeptides, are encoded by a nucleic acid that can have at least 70% sequence identity with the nucleic acid sequence of a gene encoding a mammalian SDF-1. The SDF-1 protein can also be an expression product of a genetically modified cell.

SDF-1 variants can include agonistic forms of the protein that constitutively express the functional activities of native SDF-1. Other SDF-1 variants can include those that are resistant to proteolytic cleavage, as for example, due to mutations, which alter protease target sequences. Whether a change in the amino acid sequence of a peptide results in a variant having one or more functional activities of a native SDF-1 can be readily determined by testing the variant for a native SDF-1 functional activity.

SDF-1 variants includes active fragments of an SDF-1 protein that can accelerate anal wound healing. Biologically active fragments of an SDF-1 protein include peptides comprising amino acid sequences sufficiently homologous to or derived from the amino acid sequence of a SDF-1 protein which include less amino acids than a full length SDF-1 proteins and which exhibit at least one activity of an SDF-1 protein. A biologically active portion of a SDF-1 protein can be a polypeptide that includes, 21-25, 26-30, 31-35, 36-40, 41-45, 46-50, 51-55, 56-60, or 61-65 amino acids.

### SDF-1 Expressing Polynucleotides

The SDF-1 or SDF-1 variant can also be administered to a subject using an SDF-1 expression vector to cause local expression of SDF-1 or the SDF-1 variant protein. The inventors have determined that use of expression vectors to administer SDF-1 can provide sustained SDF-1 expression that can be superior to that obtained when SDF-1 protein is administered. The SDF-1 nucleic acid that encodes the SDF-1 protein can be a native or non-native (i.e., variant) nucleic acid and be in the form of RNA or in the form of DNA (e.g., cDNA, genomic DNA, and synthetic DNA). The DNA can be double-stranded or single-stranded, and if single-stranded may be the coding (sense) strand or non-coding (anti-sense) strand. The nucleic acid coding sequence that encodes SDF-1 may be substantially similar to a nucleotide sequence of the SDF-1 gene, such as nucleotide sequence shown in SEQ ID NO: 4 and SEQ ID NO: 5. SEQ ID NO: 4 and SEQ ID NO: 5 comprise, respectively, the nucleic acid sequences for human SDF-1 and rat SDF-1 and are substantially similar to the nucleic sequences of GenBank Accession No. NM199168 and GenBank Accession No. AF189724. The nucleic acid coding sequence for SDF-1 can also be a different coding sequence which, as a result of the redundancy or degeneracy of the genetic code, encodes the same polypeptide as SEQ ID NO: 1, SEQ ID NO: 2, and SEQ ID NO: 3.

Other nucleic acid molecules that encode SDF-1 within the invention are variants of a native SDF-1, such as those that encode fragments, analogs and derivatives of native SDF-1. Such variants may be, for example, a naturally occurring allelic variant of a native SDF-1 gene, a homolog or ortholog of a native SDF-1 gene, or a non-naturally occurring variant of a native SDF-1 gene. These variants have a nucleotide sequence that differs from a native SDF-1 gene in one or more bases. For example, the nucleotide sequence of such variants can feature a deletion, addition, or substitution of one or more nucleotides of a native SDF-1 gene. Nucleic acid insertions are preferably of about 1 to 10 contiguous nucleotides, and deletions are preferably of about 1 to 10 contiguous nucleotides.

In other applications, variant SDF-1 displaying substantial changes in structure can be generated by making nucleotide substitutions that cause less than conservative changes in the encoded polypeptide. Examples of such nucleotide substitutions are those that cause changes in (a) the structure of the polypeptide backbone; (b) the charge or hydrophobicity of the polypeptide; or (c) the bulk of an amino acid side chain. Nucleotide substitutions generally expected to produce the greatest changes in protein properties are those that cause non-conservative changes in codons. Examples of codon changes that are likely to cause major changes in protein structure are those that cause substitution of (a) a hydrophilic residue (e.g., serine or threonine), for (or by) a hydrophobic residue (e.g., leucine, isoleucine, phenylalanine, valine or alanine); (b) a cysteine or proline for (or by) any other residue; (c) a residue having an electropositive side chain (e.g., lysine, arginine, or histidine), for (or by) an electronegative residue (e.g., glutamine or aspartine); or (d) a residue having a bulky side chain (e.g., phenylalanine), for (or by) one not having a side chain, (e.g., glycine).

Naturally occurring allelic variants of a native SDF-1 gene within the invention are nucleic acids isolated from mammalian tissue that have at least 70% sequence identity with a native SDF-1 gene, and encode polypeptides having structural similarity to a native SDF-1 polypeptide. Homologs of a native SDF-1 gene within the invention are nucleic acids isolated from other species that have at least 70% sequence identity with the native gene, and encode polypeptides having structural similarity to a native SDF-1 polypeptide. Public and/or proprietary nucleic acid databases can be searched to identify other nucleic acid molecules having a high percent (e.g., 70% or more) sequence identity to a native SDF-1 gene.

Non-naturally occurring SDF-1 gene variants are nucleic acids that do not occur in nature (e.g., are made by the hand of man), have at least 70% sequence identity with a native SDF-1 gene, and encode polypeptides having structural similarity to a native SDF-1 polypeptide. Examples of non-naturally occurring SDF-1 gene variants are those that encode a fragment of a native SDF-1 protein, those that hybridize to a native SDF-1 gene or a complement of to a native SDF-1 gene under stringent conditions, and those that share at least 65% sequence identity with a native SDF-1 gene or a complement of a native SDF-1 gene.

In some embodiments, natural and non-natural variants of a native SDF-1 gene have a nucleic acid sequence having more than 70% sequence identity with a native SDF-1 gene. In some embodiments, the natural or non-natural sequence variants have more than 75% sequence identity, more than 80% sequence identity, more than 85% sequence identity, more than 90% sequence identity, or more than 95% sequence identity with a native SDF-1 gene.

Nucleic acid molecules encoding a SDF-1 fusion protein may also be used in the invention. Such nucleic acids can be made by preparing a construct (e.g., an expression vector) that expresses a SDF-1 fusion protein when introduced into a suitable target cell. For example, such a construct can be made by ligating a first polynucleotide encoding a SDF-1 protein fused in frame with a second polynucleotide encoding another protein such that expression of the construct in a suitable expression system yields a fusion protein.

The nucleic acids encoding SDF-1 can be modified at the base moiety, sugar moiety, or phosphate backbone, for example, to improve stability of the molecule, hybridization, etc. The nucleic acids within the invention may additionally include other appended groups such as peptides (e.g., for targeting target cell receptors in vivo), or agents facilitating transport across the cell membrane, hybridization-triggered cleavage. To this end, the nucleic acids may be conjugated to another molecule, (e.g., a peptide), hybridization triggered cross-linking agent, transport agent, hybridization-triggered cleavage agent, etc.

### Gene Therapy

One method of introducing the agent into a target cell involves using gene therapy. Gene therapy in accordance with the present invention can be used to express SDF-1 protein from a target cell in vivo or in vitro. Gene therapy can use an expression vector including a nucleotide encoding an SDF-1 protein or protein variant. An “expression vector” (sometimes referred to as gene delivery or gene transfer “vehicle”) refers to a macromolecule or complex of molecules comprising a polynucleotide to be delivered to a target cell, either in vitro or in vivo. The polynucleotide to be delivered may comprise a coding sequence of interest in gene therapy. Vectors include, for example, viral vectors (such as adenoviruses (‘Ad’), adeno-associated viruses (AAV), and retroviruses), liposomes and other lipid-containing complexes, and other macromolecular complexes capable of mediating delivery of a polynucleotide to a target cell.

Expression vectors for use in the present invention include viral vectors, lipid based vectors and other non-viral vectors that are capable of delivering a nucleotide according to the present invention to the target cells. The expression vector can be a targeted vector, especially a targeted vector that preferentially binds to cells of proximate the wound. Viral vectors for use in the invention can include those that exhibit low toxicity to a target cell and induce production of therapeutically useful quantities of SDF-1 protein in a tissue-specific manner.

Examples of viral vectors are those derived from adenovirus (Ad) or adeno-associated virus (AAV). Both human and non-human viral vectors can be used and the recombinant viral vector can be replication-defective in humans. Where the vector is an adenovirus, the vector can comprise a polynucleotide having a promoter operably linked to a gene encoding the SDF-1 protein and is replication-defective in humans.

Other viral vectors that can be use in accordance with the present invention include herpes simplex virus (HSV)-based vectors. HSV vectors deleted of one or more immediate early genes (IE) are advantageous because they are generally non-cytotoxic, persist in a state similar to latency in the target cell, and afford efficient target cell transduction. Recombinant HSV vectors can incorporate approximately 30 kb of heterologous nucleic acid.

Retroviruses, such as C-type retroviruses and lentiviruses, can also be used as expression vectors. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and an SDF-1 nucleic acid. In methods of delivery to cells proximate the wound, it may also encode a ligand to a tissue specific receptor.

Additional retroviral vectors include replication-defective lentivirus-based vectors, such as human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells. They are also highly efficient at transducing human epithelial cells.

Alphavirus-based vectors, such as those made from semliki forest virus (SFV) and sindbis virus (SIN), might also be used in the invention. Use of alphaviruses is described in Lundstrom, K., Intervirology 43:247-257, 2000 and Perri et al., Journal of Virology 74:9802-9807, 2000. Recombinant, replication-defective alphavirus vectors are advantageous because they are capable of high-level heterologous (therapeutic) gene expression, and can infect a wide target cell range. Alphavirus replicons may be targeted to specific cell types by displaying on their virion surface a functional heterologous ligand or binding domain that would allow selective binding to target cells expressing a cognate binding partner. Alphavirus replicons may establish latency, and therefore long-term heterologous nucleic acid expression in a target cell. The replicons may also exhibit transient heterologous nucleic acid expression in the target cell.

In many of the viral vectors compatible with methods of the invention, more than one promoter can be included in the vector to allow more than one heterologous gene to be expressed by the expression vector. Further, the expression vector can comprise a sequence which encodes a signal peptide or other moiety which facilitates the secretion of a SDF-1 gene product from the target cell.

To combine advantageous properties of two viral vector systems, hybrid viral vectors may be used to deliver a SDF-1 nucleic acid to a target tissue. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the target cell genome and effect stable SDF-1 gene expression.

Other nucleotide sequence elements which facilitate expression of the SDF-1 gene and cloning of the vector are further contemplated. For example, the presence of enhancers upstream of the promoter or terminators downstream of the coding region, for example, can facilitate expression.

In accordance with another aspect of the present invention, a tissue-specific promoter can be fused to a SDF-1 gene. By fusing such tissue specific promoter within the adenoviral construct, transgene expression is limited to a particular tissue. The efficacy of gene expression and degree of specificity provided by tissue specific promoters can be determined, using the recombinant adenoviral system of the present invention.

In addition to viral vector-based methods, non-viral expression vectors may also be used to introduce a SDF-1-encoding nucleic acid into a target cell. A review of non-viral methods of gene delivery is provided in Nishikawa and Huang, Human Gene Ther. 12:861-870, 2001. An example of a non-viral gene delivery method according to the invention employs plasmid DNA to introduce a SDF-1 nucleic acid into a cell. Plasmid-based gene delivery methods are generally known in the art. Accordingly, in some embodiments, the SDF-1 expression vector is a plasmid vector. An example of a plasmid expression vector suitable for administering SDF-1 is the plasmid expression vector of SEQ ID NO: 6.

Synthetic gene transfer molecules can be designed to form multimolecular aggregates with plasmid DNA. These aggregates can be designed to bind to a target cell. Cationic amphiphiles, including lipopolyamines and cationic lipids, may be used to provide receptor-independent SDF-1 nucleic acid transfer into target cells. In addition, preformed cationic liposomes or cationic lipids may be mixed with plasmid DNA to generate cell-transfecting complexes. Methods involving cationic lipid formulations are reviewed in Feigner et al., Ann N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Methods that involve both viral and non-viral based components may be used according to the invention. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun 13:141-164, 1994.

Additionally, the SDF-1 nucleic acid can be introduced into the target cell by transfecting the target cells using electroporation techniques. Electroporation techniques are well known and can be used to facilitate transfection of cells using plasmid DNA.

Expression vectors that encode an SDF-1 polynucleotide can be delivered to the target cell in the form of an injectable preparation containing pharmaceutically acceptable carrier, such as saline, as necessary. Other pharmaceutical carriers, formulations and dosages can also be used in accordance with the present invention. In some embodiments a DNA plasmid encoding SDF-1 having the sequence of SEQ ID NO: 6 can be delivered to a target cell.

Where the target cell for an expression vector comprises a cell proximate the anal wound, the vector can be delivered by direct injection at an amount sufficient for the SDF-1 protein to be expressed to a degree that allows for highly effective therapy. By injecting the vector directly into or about the periphery of the wound, it is possible to target the vector transfection rather effectively, and to minimize loss of the recombinant vectors. This type of injection enables local transfection of a desired number of cells, especially about the wound, thereby maximizing therapeutic efficacy of gene transfer, and minimizing the possibility of an inflammatory response to viral proteins. In some embodiments the injection may be performed with a needle. In some embodiments the injection may be performed as a needle-free dermal injection.

Where the target cell is a cultured cell that is later transplanted into the anal wound (e.g., tissue graft), the vectors can be delivered by direct injection into the culture medium. A SDF-1 nucleic acid transfected into cells may be operably linked to a regulatory sequence.

The transfected target cells can then be transplanted to the wound by well known transplantation techniques, such as graft transplantation. By first transfecting the target cells in vitro and then transplanting the transfected target cells to the wound, the possibility of inflammatory response in the tissue proximate the wound is minimized compared to direct injection of the vector into cells proximate the wound.

SDF-1 can be expressed for any suitable length of time within the target cell, including transient expression and stable, long-term expression. In one aspect of the invention, the SDF-1 nucleic acid will be expressed in therapeutic amounts for a defined length of time effective to mitigate apoptosis in the cells proximate the wound and/or to promote stem cell or progenitor cell homing to the wound. This amount of time can be that amount effect to promote healing of the wound, accelerate closure of the wound, and/or inhibit scar formation.

### Combination Therapy

Other cells or agents can also be introduced into the cells to promote expression of SDF-1 from the cells. For example, agents that increase the transcription of a gene encoding SDF-1, increase the translation of an mRNA encoding SDF-1, and/or those that decrease the degradation of an mRNA encoding SDF-1 could be used to increase SDF-1 protein levels. Increasing the rate of transcription from a gene within a cell can be accomplished by introducing an exogenous promoter upstream of the gene encoding SDF-1. Enhancer elements, which facilitate expression of a heterologous gene, may also be employed.

Other agents can further include other proteins, chemokines, and cytokines, that when administered to the target cells can upregulate expression SDF-1 form the target cells. Such agents can include, for example: insulin-like growth factor (IGF)-1, which was shown to upregulate expression of SDF-1 when administered to mesenchymal stem cells (MSCs) (Circ. Res. 2008, Nov. 21; 103(11):1300-98); sonic hedgehog (Shh), which was shown to upregulate expression of SDF-1 when administered to adult fibroblasts (Nature Medicine, Volume 11, Number 11, November 23); transforming growth factor β (TGF-β); which was shown to upregulate expression of SDF-1 when administered to human peritoneal mesothelial cells (HPMCs); IL-1β, PDG-BF, VEGF, TNF-α, and PTH, which are shown to upregulate expression of SDF-1, when administered to primary human osteoblasts (HOBS) mixed marrow stromal cells (BMSCs), and human osteoblast-like cell lines (Bone, 2006, April; 38(4): 497-508); thymosin β4, which was shown to upregulate expression when administered to bone marrow cells (BMCs) (Curr. Pharm. Des. 2007; 13(31):3245-51; and hypoxia inducible factor 1α (HIF-1), which was shown to upregulate expression of SDF-1 when administered to bone marrow derived progenitor cells.

In some embodiments, at least one progenitor cell can be administered in or proximate to the anal wound together with SDF-1 or an SDF-1 expression vector. Examples progenitor cells can be selected from, but not restricted to, totipotent stem cell, pluripotent stem cell, multipotent stem cell, mesenchymal stem cell, neuronal stem cell, hematopoietic stem cell, pancreatic stem cell, cardiac stem cell, embryonic stem cell, embryonic germ cell, neural crest stem cell, kidney stem cell, hepatic stem cell, lung stem cell, hemangioblast cell, and endothelial progenitor cell. Additional examples of progenitor cells can be selected from, but not restricted to, de-differentiated chondrogenic cells, myogenic cells, osteogenic cells, tendogenic cells, ligamentogenic cells, adipogenic cells, and dermatogenic cells.

### Administration of SDF-1

The present invention includes administering a therapeutically effective amount of a stromal cell-derived factor-1 (SDF-1) protein or protein variant, or an SDF-1 or SDF-1 variant expression vector in or proximate to an anal wound. In some embodiments, the wound healing composition includes a pharmaceutically acceptable carrier to facilitate administration. The active agent (e.g., the SDF-1 protein or protein variant, or an SDF-1 or SDF-1 variant expression vector) is preferably utilized together with one or more pharmaceutically acceptable carrier(s) and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect (i.e., wound healing), and in a quantity appropriate to achieve the desired daily dose. The SDF-1 may also be covalently attached to a protein carrier, such as albumin, so as to decrease metabolic clearance of the peptides.

In some embodiments, the SDF-1 or SDF-1 variant expression vector is injected into the anal wound or an area proximate to the anal wound. In some embodiments, proximate administration is administration within 1 inch of the wound, or within 2 inches of the wound. Typically, the SDF-1 will be suspended in a sterile saline solution for therapeutic uses. The pharmaceutical compositions may alternatively be formulated to provide sustained release of SDF-1 locally. Numerous suitable drug delivery systems are known and include, e.g., implantable drug release systems, hydrogels, hydroxymethylcellulose, microcapsules, liposomes, microemulsions, microspheres, and the like. Sustained release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid. The rate of release of the active ingredient(s) from such a matrix depends upon the molecular weight of the active agent, the amount of the active agent within the matrix, and other factors known to those skilled in the art.

It will be apparent to those of ordinary skill in the art that the therapeutically effective amount of SDF-1 will depend, inter alia upon the administration schedule, the unit dose of molecule administered, whether the peptide or expression vector is administered in combination with other therapeutic agents, the immune status and health of the patient, the therapeutic activity of the peptide administered and the judgment of the treating physician.

The SDF-1 can be dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those skilled in the art. In addition, if desired, the composition can contain minor amounts of auxiliary substances such as wetting or emulsifying agents, pH buffering agents.

In some embodiments, a single dose of SDF-1 or an SDF-1 expression vector is administered. However, in other embodiments, the SDF-1 protein or protein variant administered repeatedly or continuously over a significant period of time. This can be achieved either through repeated administration, or through use of a sustained-release formulation.

In certain embodiments, liposomes and/or nanoparticles may also be employed to administer the SDF-1. The formation and use of liposomes is generally known to those of skill in the art. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 angstroms, containing an aqueous solution in the core.

In another aspect of the present invention, the SDF-1 or SDF-1 protein variant can be formulated for topical administration to treat anal wounds. Accordingly, in some embodiments, the SDF-1 protein or protein variant, or SDF-1 or SDF-1 variant expression vector is administered as a topical formulation Topical delivery systems may be used to administer topical formulations of the present invention. Formulations for topical administration to the anal area can include ointments, creams, gels, and pastes comprising SDF-1 or SDF-1 agent to be administered in a pharmaceutically acceptable carrier. Topical formulations can be prepared using oleaginous or water-soluble ointment bases, as is well known to those in the art. For example, these formulations may include vegetable oils, animal fats, and more preferably semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin, and glyceryl monostearate. Various water-soluble ointment bases may also be used including, for example, glycol ethers and derivatives, polyethylene glycols, polyoxyl 40 stearate, and polysorbates.

In another aspect of the invention, SDF-1 or SDF-1 expression vector can be provided in and/or on a substrate, solid support, and/or wound dressing for delivery of the SDF-1 or agent to the anal wound. As used herein, the term “substrate,” or “solid support” and “wound dressing” refer broadly to any substrate when prepared for, and applied to, a wound for protection, absorbance, drainage, etc. The present invention may include any one of the numerous types of substrates and/or backings that are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (non-woven composites of fibers from calcium alginate), and cellophane (cellulose with a plasticizer). The shape and size of the anal wound may be determined and the wound dressing customized for the exact site based on the measurements provided for the wound. As wound sites can vary in terms of mechanical strength, thickness, sensitivity, etc., the substrate can be molded to specifically address the mechanical and/or other needs of the site.

In one example, the substrate can be a bioresorbable implant that includes a polymeric matrix and the SDF-1 or SDF-1 expression vector dispersed in the matrix. The polymeric matrix may be in the form of a membrane, sponge, gel, scaffold, or any other desirable configuration. The polymeric matrix can be formed from biodegradable polymer. It will be appreciated, however, that the polymeric matrix may additionally comprise an inorganic or organic composite. The polymeric matrix can comprise any one or combination of known materials including, for example, chitosan, poly(ethylene oxide), poly (lactic acid), poly(acrylic acid), poly(vinyl alcohol), poly(urethane), poly(N-isopropyl acrylamide), poly(vinyl pyrrolidone) (PVP), poly (methacrylic acid), poly(p-styrene carboxylic acid), poly(p-styrenesulfonic acid), poly(vinylsulfonicacid), poly(ethyleneimine), poly(vinylamine), poly(anhydride), poly(L-lysine), poly(L-glutamic acid), poly(gamma-glutamic acid), poly(caprolactone), polylactide, poly(ethylene), poly(propylene), poly(glycolide), poly(lactide-co-glycolide), poly(amide), poly(hydroxylacid), poly(sulfone), poly(amine), poly(saccharide), poly(HEMA), poly(anhydride), collagen, gelatin, glycosaminoglycans (GAG), poly (hyaluronic acid), poly(sodium alginate), alginate, hyaluronan, agarose, polyhydroxybutyrate (PHB), and the like. In some embodiments, the topical formulation comprises a hydrogel scaffold.

It will be appreciated that one having ordinary skill in the art may create a polymeric matrix of any desirable configuration, structure, or density. By varying polymer concentration, solvent concentration, heating temperature, reaction time, and other parameters, for example, one having ordinary skill in the art can create a polymeric matrix with any desired physical characteristic(s). For example, the polymeric matrix may be formed into a sponge-like structure of various densities. The polymeric matrix may also be formed into a membrane or sheet which could then be wrapped around or otherwise shaped to a wound. The polymeric matrix may also be configured as a gel, mesh, plate, screw, plug, or rod. Any conceivable shape or form of the polymeric matrix is within the scope of the present invention. In an example of the present invention, the polymeric matrix can comprise a alginate matrix.

The polymeric matrix of the present invention may be seeded with at least one progenitor cell and the SDF-1 or SDF-1 expression vector The SDF-1 or SDF-1 expression vector can be dispersed in matrix and/or expressed from the seeded progenitor cell. Progenitor cells can include autologous cells; however, it will be appreciated that xenogeneic, allogeneic, or syngeneic cells may also be used. Where the cells are not autologous, it may be desirable to administer immunosuppressive agents in order to minimize immunorejection. The progenitor cells employed may be primary cells, explants, or cell lines, and may be dividing or non-dividing cells. Progenitor cells may be expanded ex vivo prior to introduction into the polymeric matrix. Autologous cells are preferably expanded in this way if a sufficient number of viable cells cannot be harvested from the host.

Another aspect of the invention provides a topical formulation for treating an anal or sphincter wound, comprising a topical pharmaceutical carrier and an SDF-1 protein or protein variant, or an SDF-1 or SDF-1 variant expression vector. The topical formulation can include any of the topical formulations described herein. For example, in some embodiments, the pharmaceutical carrier of the topical formulation comprises a hydrogen scaffold. When a polymeric matrix is included as a topical pharmaceutical carrier, in some embodiments the topical formulation further comprises progenitor cells such as mesenchymal stem cells.

The SDF-1 protein or protein variant, or the SDF-1 or the SDF-1 or SDF-1 expression vector can include any of the proteins, protein variants, or expression vectors described herein. For example, in some embodiments, the SDF-1 protein comprises SEQ ID NO: 1. In some embodiment, the expression vector is a viral vector, while in other embodiments the expression vector is a non-viral expression vector. For example, in some embodiments the non-viral SDF-1 expression vector is a plasmid vector, such as the SDF-1 expression vector comprising SEQ ID NO: 6.

Examples have been included to more clearly describe particular embodiments of the invention. However, there are a wide variety of other embodiments within the scope of the present invention, which should not be limited to the particular examples provided herein.

### EXAMPLES

### Example 1: Regenerating the Anal Sphincter: Cytokines, Stem Cells, or Both?

We have demonstrated upregulation of two cytokines, stromal derived factor 1 (SDF-1) and monocyte chemotactic protein 3, after an acute anal sphincter injury. Salcedo et al., J Colorectal Dis., 26:1577-1581 (2011). In addition, we have shown that these chemokines are downregulated over 3 weeks, which can result in suboptimal healing by fibrosis. SDF-1, along with its receptor, CXCR4, is responsible for chemotaxis (Aiuti et al., J Exp Med., 185:111-120 (1997)), angiogenesis (Peled et al., Science, 283:845-848 (1999)), and transendothelial migration of CD34+ cells. Peled et al., Blood, 95, 3289-3296 (2000) This cytokine has been in clinical trials for chronic heart failure, chronic limb ischemia, and wound healing. The study evaluated regeneration of a large defect of the anal sphincter at a time remote from injury using a plasmid encoding for SDF-1 alone or in conjunction with exogenously administered mesenchymal stem cells (MSCs) within and without a gelatin scaffold at a time distant from injury.

### Materials and Methods

This study used weight- and age-matched virgin female Sprague Dawley rats (Charles River Laboratories, Wilmington, Mass.; 250 to 300 g). We investigated the expression of a luciferase plasmid (SDF-1 replaced by luciferase) after a large gluteus muscle injury. This experiment also determined the dose of the plasmid to be used for the next phase. Next, we evaluated the effect of the plasmid on a chronic large anal sphincter defect and evaluated resting pressure and histology as outcomes.

### Luciferase-Encoded Plasmid and SDF-1-Encoded Plasmid

The luciferase-encoded plasmid (pLUC) and SDF-1-encoded plasmid (pSDF-1) were obtained from Juventas Therapeutics, Inc (Cleveland, Ohio). The two recombinant DNA plasmids are grown and purified from Escherichia coli in a guanosine 5′-monophosphate manufacturing facility. The pLUC plasmid has luciferase replacing SDF-1 and was used to evaluate plasmid expression. Both pLUC and pSDF-1 for use in this study were prepared as 2 mg/mL in sucrose solution.

### Evaluation of Plasmid Expression and Determination of the Dose of Plasmid

Sprague Dawley rats underwent anesthesia with ketamine (100 mg/kg) and xylazine (10 mg/kg) given intraperitoneally. An incision was made exposing the gluteus major and minor muscles. A defect was created by excising muscle measuring 1.0×1.0×0.3 cm, 0.5 g in weight, in all of the animals. The wound was closed after an intramuscular injection into the wound wall at 4 points of either 100 μg (50 μL) of pLUC (LUC100, n=6), 200 μg (100 μL) of pLUC (LUC200, n=6), or 100μ: of or phosphate-buffered saline solution (n=6).

### In Vivo Bioluminescence Imaging

pLUC expression at the site of injury was evaluated by in vivo bioluminescence using the IVIS Lumina Series II (PerkinElmer, Waltham, Mass.). On days 1, 3, 5, 7, 9, 11, 13, 15, and 30 after intramuscular injection of pLUC and injection with D-luciferin (PerkinElmer) intraperitoneally in 100 mg/kg in phosphate buffered saline solution (20 mg/mL), 3 minutes before carrying out the in vivo bioluminescence imaging, the measurement was taken. The signal strength was quantified (photons per second per cm² per radiation) for analysis of pLUC expression.

### T-Gelatin Hydrogel Scaffold Preparation

The hydrogel scaffold was provided by the laboratory of Dr Calabro, which has a novel cross-linking method. This type-1, collagen-based scaffold contains tyramine-substituted gelatin (T-gelatin), which was enzymatically crosslinked in the presence of hydrogen peroxide.

Evaluating Outcomes after an Anal Sphincter Injury

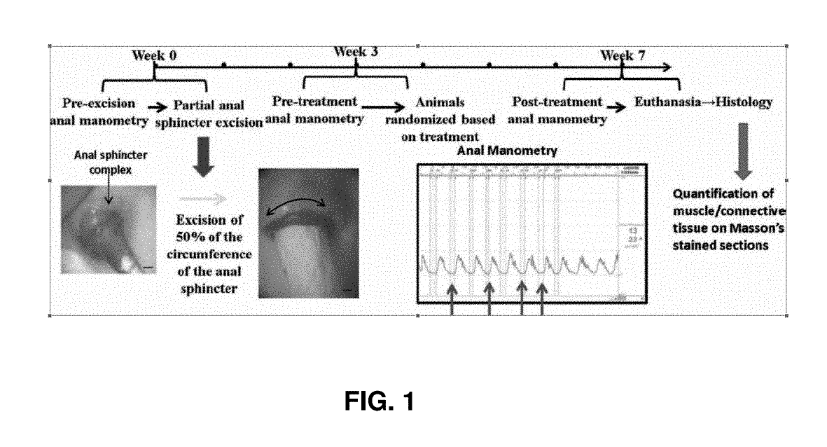

Thirty-two Sprague Dawley rats underwent an excision of the ventral portion of 50% circumference of the anal sphincter under ketamine/xylazine intraperitoneal anesthesia, as per our previous protocol. Salcedo et al., Stem Cells Transl Med., 3:760-767 (2014) The surgery was carried out by a single operator. The animals were allowed to recover for 3 weeks (W3) when they were randomly assigned to 4 groups, as follows: 1) internal anal (IA), no treatment; 2) pSDF-1, 100 μg of pSDF-1 injected at the site of the defect; 3) pSDF-1+MSC, pSDF-1 and MSCs injected at the site of the defect; and 4) pSDF-1+S & MSC, pSDF-1 injected at the site of the defect 3 days before the injection of a gelatin scaffold mixed with MSC (n=8 per group). All of the groups were evaluated 4 weeks after treatment (W7; FIG. 1). All of the groups using MSCs used 800,000 cells.

### MSC Harvesting, Culture, and Identification

The MSC harvesting and sorting protocol was conducted as described in our previous study. Salcedo et al., Stem Cells Transl Med., 3:760-767 (2014) In brief, bone marrow from Sprague Dawley rats was harvested from the tibial and femoral bones. MSC culture medium was made from Dulbecco Modified Eagle Medium (Invitrogen, Carlsbad, Calif.) with 12.5% fetal bovine serum (Gibco, Thermo Fisher Scientific, Waltham, Mass.) and 1.0% antibiotic and antimycotic solution (Gibco). When 70% to 80% confluence was reached, the cells were passaged using Trypsin-EDTA (Gibco). For sorting, cells were incubated with intracellular adhesion molecule 1 antibody (Abcam, Cambridge, United Kingdom), including 5 μL/10⁶ cells for 30 minutes at room temperature. The sorting of intracellular adhesion molecule 1−positive MSC was performed with an LSRII flow cytometer (Becton Dickinson, Franklin Lakes, N.J.). Sorted MSC were cultured until passage 10 to 12 for study.

MSC lineage analysis was performed before transduction using a rat MSC functional identification kit (R&D Systems, Inc, Minneapolis, Minn.), according to the manufacturer's instructions Immunocytochemistry was performed using antibodies to fatty acid binding protein 4, aggrecan and osteocalcin, to define mature phenotypes of adipocytes, chondrocytes, and osteocytes.

### Anal Manometry

Anal sphincter function was assessed preinjury and before and 4 weeks after treatment, as in our previous study (FIG. 1). Salcedo et al., Stem Cells Transl Med., 3:760-767 (2014) Under anesthesia, as described previously, a 7-FT-Doc air-charged catheter (Laborie Medical Technologies, Mississauga, Ontario, Canada) was inserted into the anal canal, and the Goby anorectal manometry system (Laborie Medical Technologies) was used for recording pressures. Resting pressure was determined as a stable baseline pressure recording. Eight typical pressure waves were collected for analysis at preinjury (W0), just before treatment (W3), and 4 weeks after treatment (W7). The mean of the pressures in each animal was used as the outcome variable for additional group analysis.

### Histology

Histologic assessment was done at W7, 4 weeks after treatment, as described previously. Sun et al., Dis Colon Rectum., 59:434-442 (2016) Briefly, after euthanasia, the anal canal was harvested, formalin fixed, and paraffin embedded before being sectioned (5 μm). Masson trichrome staining was performed on serial sections separated by ≥100 μm throughout the anal sphincter complex of each specimen. The sections were scanned using the Leica SCN400 Slide Scanner (Leica Microsystems Inc, Buffalo Grove, Ill.) at 20× magnification before being viewed by a blinded observer under a bright-field microscope. Quantification of muscle and connective tissue was performed using the Image-Pro Plus 7.0 software (Media Cybernetics, Inc, Rockville, Md.).

The muscle and connective tissue at the site of the defect were calculated as a percentage of the combined muscle and connective tissue. The intact muscle and connective tissue were similarly calculated at the site of the uninjured area in the same section. The two muscle and connective tissue percentages were then compared and were expressed as a ratio.

### Statistical Analysis

Bioluminescence data and anal pressure measurements of different groups over time were analyzed by 2-way ANOVA followed by Tukey post hoc test using the SigmaPlot 11.0 (Systat Software Inc, San Jose, Calif.). The comparison of pressures between W3 (pretreatment) and W7 (posttreatment) and the results of histological quantification were calculated using 1-way ANOVA, followed by the Tukey post hoc test in SigmaPlot 11. All of the results are expressed as mean±SEM. A p value of <0.05 was regarded as indicating a statistically significant difference in all of the comparisons.

### Results

No mortality or morbidity was noted after treatment during the course of this experiment.

### Luciferase Plasmid Expression in Gluteal Wound

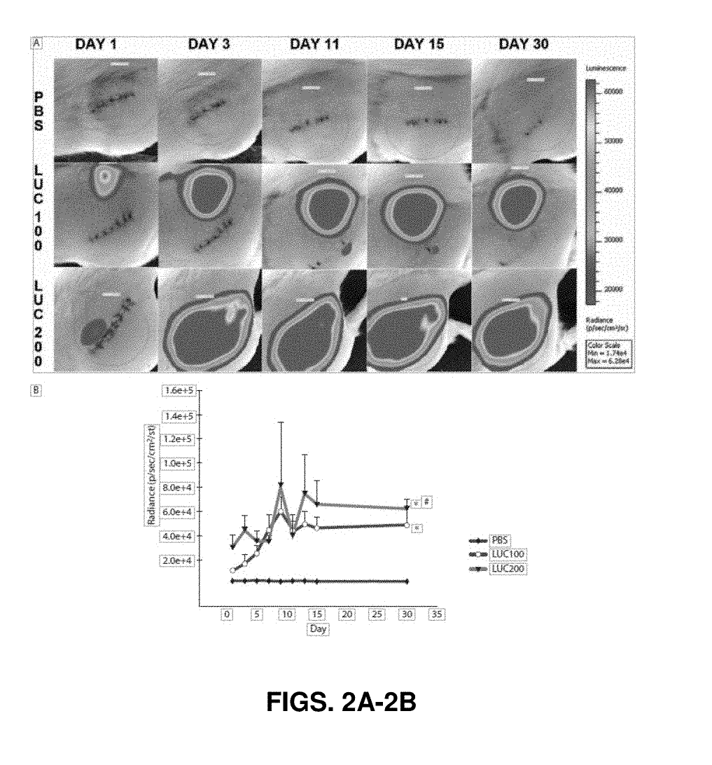

In vivo pLUC expression was noted as a strong bioluminescence signal at day 3, which peaked between days 11 and 15 and was noted up to 30 days post-injection in the gluteal wound (FIG. 2A). Both doses, LUC100 and LUC200, showed a significantly higher bioluminescence than phosphate-buffered saline solution (p<0.05). There was significantly higher bioluminescence in the LUC200 group than either LUC100 or phosphate-buffered saline solution (p<0.05; FIG. 2B). On the basis of these results, both doses induced protein expression in the muscle for an extended time with no benefit of a larger dose. The lower dose of 100 μg was therefore chosen for the anal sphincter experiment.

### MSC Identification Analysis

Multipotency verification was performed on MSC, which confirmed the differentiation capability of the expanded MSC into chondrogenic, osteogenic, and adipogenic cells.

### Functional and Histological Analysis on Anal Sphincter

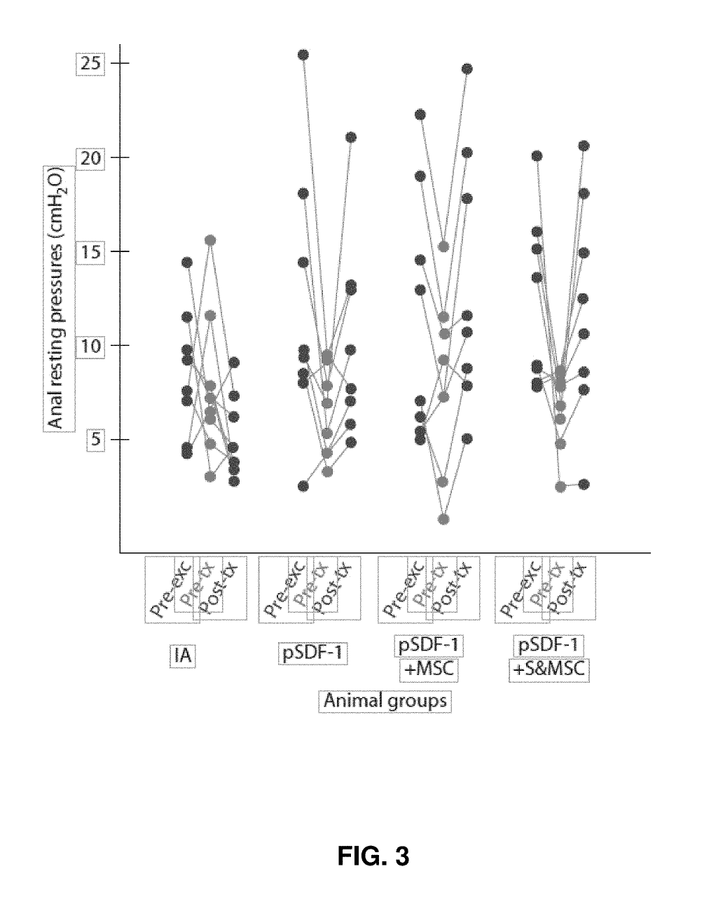

Before the injury (W0), resting pressure among the four groups was similar (Table 1). The resting pressure in all of the groups declined after injury (W3) and was not significantly different between groups. At the post-injury time point of W7, there was no significant difference noted when pressures were compared before and after treatment in the IA group. However, there was a significant increase in anal resting pressure from pretreatment to the post-treatment time point in the group receiving pSDF-1 alone and the group with pSDF-1 injection 3 days before the injection of a gelatin scaffold mixture with MSC. These 2 groups also reached pre-excision anal resting pressures. However, no significant difference was noted in anal pressures before/after treatment in the pSDF-1+MSC group (Table 1 and FIG. 3).

TABLE 1
Results of functional outcome and histology

| | | | | |
|---|---|---|---|---|
| | | | pSDF- | |
| IA | pSDF-1 | pSDF-1 + MSC | 1 + S&MSC | p value |

Anal Pressures

| | | | | | |
|---|---|---|---|---|---|
| Pre-excision | 8.6 ± 1.22 | 12.3 ± 1.61 | 11.6 ± 2.35 | 12.0 ± 2.51 | IA vs. pSDF-1 + MSC p = 0.4 |
| (W0) | | | | | IA vs. pSDF-1 + S&MSC p = 0.6 |
| | | | | | IA vs. pSDF-1 p = 0.5 |
| | | | | | pSDF-1 vs. pSDF-1 + MSC p = 1.0 |
| | | | | | pSDF-1 vs. pSDF-1 + S&MSC p = 1.0 |
| | | | | | pSDF-1 + MSC vs. pSDF-1 + S&MSC p = 1.0 |
| Pre-treatment | 7.9 ± 0.97 | 6.7 ± 0.44 | 8.1 ± 1.02 | 6.3 ± 0.55 | IA vs. pSDF-1 p = 1.0 |
| (W3) | | | | | IA vs. pSDF-1 + MSC p = 1.0 |
| | | | | | IA vs. pSDF-1 + S&MSC p = 0.9 |
| | | | | | pSDF-1 vs. pSDF-1 + MSC p = 0.9 |
| | | | | | pSDF-1 vs. pSDF-1 + S&MSC p = 0.9 |
| | | | | | pSDF-1 + MSC vs. pSDF-1 + S&MSC p = 1.0 |
| Post- | 5.2 ± 0.76 | 12.0 ± 2.08 | 1.4 ± 2.42 | 10.3 ± 1.88 | Comparison before (W3) and after |
| treatment | | | | | treatment (W7) |
| (W7) | | | | | IA p = 0.17 |
| | | | | | pSDF-1 p = 0.03 |
| | | | | | pSDF-1 + MSC p = 0.03 |
| | | | | | PSDF-1 + S&MSC p = 0.4 |
| | | | | | IA vs. pSDF-1 p = 0.04 |
| | | | | | IA vs. pSDF-1 + MSC p = 0.008 |
| | | | | | IA vs. pSDF-1 + S&MSC p = 0.17 |
| | | | | | pSDF-1 vs. pSDF-1 + MSC p = 1.0 |
| Change in | −2.6 ± 1.47 | 5.3 ± 1.80 | 3.7 ± 1.65 | 5.3 ± 1.50 | Compared to IA |
| pressures | | | | | pSDF-1 p = 0.009 |
| (cm of H₂O) | | | | | PSDF-1 + MSC p = 0.009 |
| | | | | | PSDF-1 + S&MSC p = 0.47 |

Histology

| | | | | | |
|---|---|---|---|---|---|
| Quantification | 0.87 ± 0.03 | 1:09 ± 0.16 | 1.0 ± 0.25 | 0.93 ± 0.31 | IA vs. pSDF-1 p = 0.18 |
| of muscle | | | | | IA vs. pSDF-1 + MSC p = 0.01 |
| | | | | | IA vs. pSDF-1 + S&MSC p = 0.4 |
| | | | | | pSDF-1 vs. pSDF-1 + MSC p = 0.6 |
| | | | | | pSDF-1 vs. pSDF-1 + S&MSC p = 1.0 |
| | | | | | pSDF-1 + MSC vs. pSDF-1 + S&MSC p = 0.4 |
| Quantification | 1.4 ± 0.05 | 1.16 ± 0.04 | 1.09 ± 0.06 | 1.19 ± 0.06 | IA vs. pSDF-1 p = 0.03 |
| of connective | | | | | IA vs. pSDF-1 + S&MSC, p = 0.07 |
| tissue | | | | | pSDF-1 vs. pSDF-1 + MSC p = 0.8 |
| | | | | | pSDF-1 vs. pSDF-1 + S&MSC p = 1.0 |
| | | | | | pSDF-1 + MSC vs. pSDF-1 + S&MSC p = 0.6 |

When comparing the change in pressure from W3 to W7, there was a significant increase in all 3 of the groups receiving the plasmid compared with the IA group (−2.60±1.47 cmH₂O), which decreased from the pretreatment baseline (pSDF-1: 5.30±1.80 cmH₂O, p=0.009; pSDF-1+MSC: 3.70±1.65 cmH₂O, p=0.047; pSDF-1+S & MSC: 5.30±1.50 cmH₂O, p=0.009). However, on intragroup comparison of the groups receiving the plasmid, there was no significant difference in the change in pressure from baseline (pSDF-1 vs pSDF-1+MSC: p=0.06; pSDF-1 vs pSDF-1+S & MSC: p=0.1; pSDF-1+MSC vs pSDF-1+S & MSC: p=0.09; Table 1).

### Histology

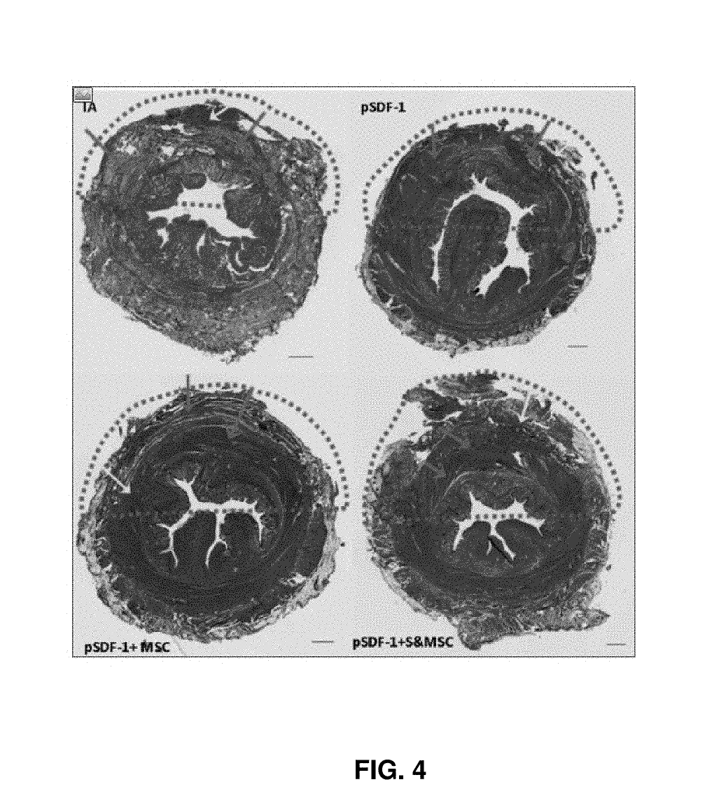

All of the groups receiving the plasmid showed filling of the defect with muscle fibers, with the pSDF-1+MSC group showing the greatest organization of muscle fibers. The area of the defect in the IA group, however, showed patchy filling of the defect with a disorganized architecture. The pSDF-1+MSC group was more comparable in histology to uninjured muscle than both pSDF-1 and pSDF-1+S & MSC groups (FIG. 4).

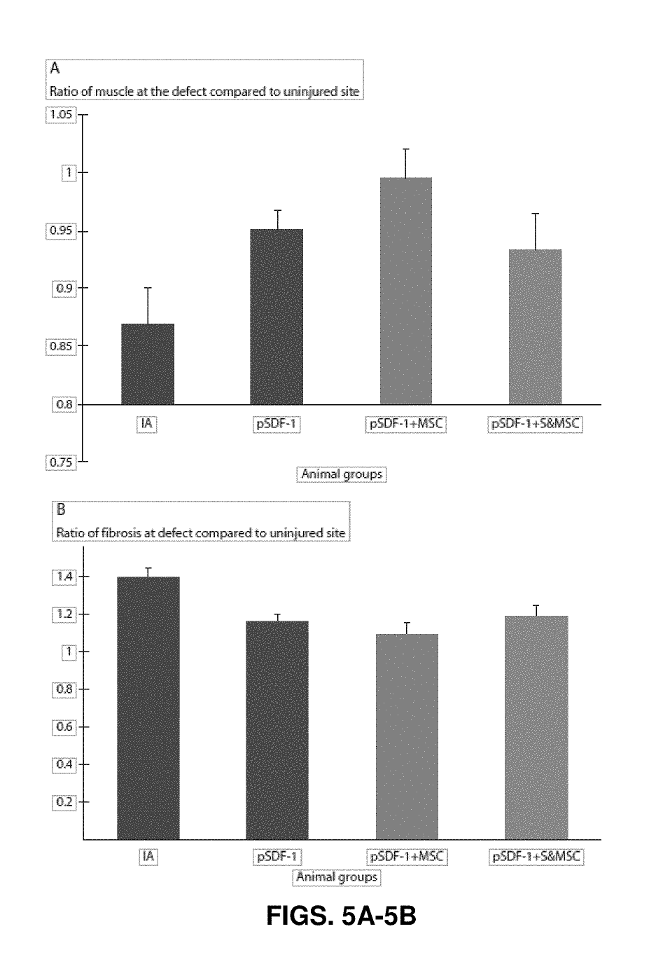

Quantification of the muscle at the site of injury revealed that the pSDF-1+MSC group had a significantly greater muscle ratio compared with the IA group (Table 1 and FIG. 5A), whereas no significant difference in muscle quantification was noted between the other groups. Quantification of connective tissue showed that significantly less fibrosis (Table 1) was seen in groups pSDF-1 and pSDF-1+MSC compared with the IA group, whereas there was no significant difference noted between the other groups (FIG. 5B).

### Discussion

Fecal incontinence attributed to childbirth injury or trauma results in defects that can be large. We chose to evaluate a large defect, and we used different treatment options to see which treatment fills the entire defect. In the event that the plasmid and plasmid with MSC groups did not heal large defects, we added a scaffold to deliver treatment to large defects. Lu et al., Urology, 61:1285-1291 (2003); Rahman et al., J Biomed Mater Res B Appl Biomater, 101:648-655 (2013)

This is the first study that evaluates regeneration in a chronic anal sphincter injury with no ongoing active inflammation using a cytokine as an agent to mimic the milieu of an acute injury. The repair that ensues when the plasmid is injected without exogenous cells is presumed to be attributed to migration of innate stem cells. Chiriac et al., J Cardiovasc Transl Res., 3:674-682 (2010) Local SDF-1 can increase homing of bone marrow-derived cells to sites of traumatic injury in a study that tagged MSCs to reach a contused and non-contused lung. Hannoush et al., J Trauma., 71:283-289 (2011)

The increased anal pressures and histological evidence of repair in the group treated with the plasmid alone establishes the fact that the cytokine facilitates initializing and sustaining the repair process. The group that included MSCs did not significantly enhance the repair process over the plasmid alone, thereby indicating that a non-cellular therapy alone may suffice. We did not observe any untoward effects of the plasmid or its concurrent use with MSCs or a scaffold.

The microenvironment after an acute injury is conducive to repair, although cell signaling wanes over time, preventing complete repair and regeneration. Salcedo et al., J Colorectal Dis., 26:1577-1581 (2011) However, at a time remote from injury, there is no active process of repair. This area is devoid of factors that chemoattract stem cells or activate quiescent local stem cells. The study by Bisson et al. has shown that cells injected at a site where there is no ongoing inflammatory process do not contribute to the process of repair, and no regeneration occurred at the site of injection within a normal muscle. Bisson et al., Cell Transplant., 24:277-286 (2015). We have shown in this study that exogenously administered SDF-1 delivered as a nonviral plasmid is expressed in the tissues after injection maximally at 3 days. Delivering MSCs during this time allows the MSCs to be retained at the target site, similar to delivery of stem cells after an acute injury, and, because MSCs are expressed up to 11 days, the process of repair is initiated and achieves completion.

The advantage of using nonviral vectors is because of their biosafety, low immunogenicity, and multiple dosing, if needed. Yin et al., Nat Rev Genet., 15, 541-555 (2014). Nonviral vectors have been shown to have local effects in tissues despite not being as efficient as viral vectors. Pickering et al., Circulation, 89:13-21 (1994) SDF-1 as a plasmid has been shown to be expressed in the infarct border zone and to improve cardiac function 1 month after delivery. Sundararaman et al., Gene Ther., 18:867-873 (2011) The SDF-1 plasmid also has few adverse effects, and its safety has been documented previously. Penn et al., Gene Ther., 19:583-587 (2012) Local injections of SDF-, along with MSC sheets, have been reported to increase bone union in dogs. Chen et al., Cell Transplant, 25(10):1801-1817 (2016) Nano-sized SDF-1 liposomes have also been reported to heal mouse diabetic wounds. Olekson et al., Wound Repair Regen., 23:711-723 (2015).

The strength of this study lies in the fact that we have evaluated regeneration in a model of a chronic injury. In addition, we have evidence of muscle at the site of injury in the groups receiving the plasmid, and we have quantified the muscle and shown that the treatment groups had much better functional and histological outcomes than the animals that did not receive any intervention. Furthermore, we have also corroborated the histology findings with changes in resting anal pressure. Finally, we have a cellular and a non-cellular treatment option, which may have clinical applications.

All of the preclinical animal research involving anal sphincter regeneration have used the model of an acute sphincter injury and treatment to evaluate different cell-based therapies. Aghaee-Afshar et al demonstrated an increase in EMG and healing of the defect with muscle. Aghaee-Afshar et al., Dis Colon Rectum, 52:1753-1761 (2009) Kajbafzadeh et al. reported a decrease in anal pressures by 87% and an increase to 74% after muscle progenitor cell transplant in a rabbit model. Kajbafzadeh et al., Dis Colon Rectum., 53:1415-1421 (2010) Likewise, Lorenzi et al. demonstrated an increase in the muscle fraction area in the groups treated with MSC and also an increase in EMG contraction compared with control but not with the sham. Lorenzi et al., Dis Colon Rectum, 51:411-420 (2008) Pathi et al. evaluated neurophysiological studies 21 days after injury and reported full recovery in rats treated with direct MSC injection and partial recovery with those treated with an intravenous injection. Pathi et al., Obstet Gynecol., 119:134-144 (2012). Fitzwater et al. did not demonstrate an increase in muscle volume between cell- and sham-treated animals. Fitzwater et al., Int Urogynecol J., 26:251-256 (2015). They reported histological findings but did not quantify the muscle mass. They also reported no beneficial effect in animals where the cut ends were not repaired. We have demonstrated that the sphincterotomy in rats heals at 4 weeks and therefore have used a model that excises part of the anal sphincter, which does not heal spontaneously. Salcedo et al., Dis Colon Rectum., 53:1209-1217 (2010).

A few studies have evaluated stem cells in a scaffold. Montoya et al. reported an increase in histology and contractile forces in a rat model of anal sphincter transection and 2 weeks later re-exposing the muscle and treating with a hydrogel matrix with a commercial myoblast cell line. Montoya et al., Int Urogynecol J., 26:893-904 (2015) Oh et al. used a dog model of anal sphincter excision and treatment with polycaprolactone beads as a bulking agent with myoblasts and have reported an increase in resting and contractile pressures in in vitro studies. Oh et al. Dis Colon Rectum, 58:517-525 (2015) Using scaffold as a delivery for stem cells has some evidence for the improvement of outcomes in vitro similar to our in vivo results.

### Conclusion

This example successfully demonstrated that the area of a large defect of the anal sphincter can be regenerated long after the injury in a rat model. Treating the area of intended repair with a cytokine, that is, a plasmid encoding for SDF-1 with and without MSCs, achieves increased anal sphincter pressures and has demonstrable evidence of regenerating muscle at a midterm time point of 4 weeks. This indicates that innate and exogenously administered stem cells can be chemoattracted using a cytokine to effectively mediate a repair of a chronic anal sphincter injury. Future studies should focus on later time points and histological differentiation of the regenerated muscle.

### Example 2: Stromal Cell-Derived Factor 1 Plasmid Regenerates Both Smooth and Skeletal Muscle after Anal Sphincter Injury in the Long Term

In treatment of fecal incontinence, an ideal regenerative therapy would regenerate muscle in the setting of a chronic injury and would have a sustained regenerative effect that is also functionally effective. To achieve this goal, we have evaluated the tissue environment that occurs after an acute injury and have reported on the cytokines, stromal cell-derived factor 1 (SDF-1) and monocytic chemotactic protein 3, which are upregulated after injury and decline 3 weeks later. Salcedo et al., Int J Colorectal Dis., 26:1577-1581 (2011). We have also evaluated the regenerative potential of bone marrow-derived mesenchymal stem cells (MSCs) in the setting of an acute injury and have postulated that regeneration may be the result of paracrine effects of the MSCs, as we have not shown evidence of survival of exogenously implanted MSCs. Salcedo et al., Stem Cells Transl Med., 3:760-767 (2014) In the chronic setting we have shown early regeneration of muscle four weeks after treatment with a plasmid encoding for SDF-1 with and without MSCs given 3 weeks after a large anal sphincter defect. Sun et al., Dis Colon Rectum, 60:416-425 (2017).

In this study we aimed to evaluate whether this effect is sustained and to study tissue morphology 8 weeks after treatment. We hypothesized that the plasmid encoding for SDF-1 regenerates both smooth and skeletal muscles with sustained functional improvement in a rat model of a chronic anal sphincter injury 8 weeks after treatment.

### Materials and Methods

### SDF-1-Encoded Nonviral Plasmid

The SDF-1-encoded plasmid was obtained from Juventas Therapeutics, Inc (Cleveland, Ohio), as was used in the previous study. Sun et al., Dis Colon Rectum. 59: 434-442 (2016). This study used the SDF-1 plasmid in dextrose solution with a concentration of 2 mg/mL.

### MSC Harvesting, Culture, and Identification

The harvesting and sorting protocol for rat bone marrow-derived MSCs was followed as per our previous study. Sun et al., ibid. In brief, the tibia and femoral bone marrow was harvested and the cells were cultured in MSC culture medium made from Dulbecco's Modified Eagle Medium (Invitrogen, Carlsbad, Calif.), 12.5% fetal bovine serum (GIBCO, Invitrogen), and 1% antibiotic and antimycotic solution (GIBCO, Invitrogen). The cells were passaged when they reached 70% to 80% confluence using trypsin-EDTA (GIBCO, Invitrogen). Intracellular adhesion molecule 1 antibody (Abcam, Cambridge, United Kingdom) was used for cell sorting with the concentration of 5 μl/10⁶ cells for 30 minutes at room temperature. The sorting of intracellular adhesion molecule 1-positive MSCs was performed with an LSRII flow cytometer (BD, Franklin Lakes, N.J.). MSCs at passage 8 to 12 were used for this study.

MSC lineage analysis was performed using a rat MSC functional identification kit (R&D Systems, Inc, Minneapolis, Minn.) following the manufacturer's instruction. Immunocytochemistry was used to define mature phenotypes of adipocytes, chondrocytes, and osteocytes with antibodies to fatty acid binding protein 4, aggrecan, and osteocalcin, separately.

### T-Gelatin Hydrogel Scaffold Preparation

The type-1 collagen-based scaffold was obtained courtesy of Dr. Anthony Calabro (Cleveland Clinic Lerner Research Institute). It contains tyramine-substituted gelatin (TGelatin) using a novel enzymatic cross-linking method in the presence of hydrogen peroxide, as used in our previous study. Bruggeman et al., Cell Mol Bioeng., 5: 194-204 (2012).

### Rat Model of a Chronic Large Anal Sphincter Defect

We used age- and weight-matched virgin female Sprague-Dawley rats (250 to 300 g) for this study. The animals were anesthetized with a ketamine (100 mg/kg intraperitoneal) and xylazine (10 mg/kg intraperitoneal) mixture before undergoing an excision of 50% of the circumference of the ventral portion of the anal sphincter. All of the procedures were carried out by a single operator. The animals were allowed to recover for 3 weeks. At this 3-week time point they were randomly allocated to 4 groups, as follows: group IA included injury without any treatment; group P included 50 μL of SDF-1 plasmid solution (100 μg) injected at the ends of the defect; group P+MSC included MSCs injected at the ends of the defect 3 days after injection of SDF-1 plasmid; and group P+S&MSC included gelatin scaffold and MSC mixture injected into the site of the defect 3 days after injection of SDF-1 plasmid in the same area. All of the MSC-treated animals received 800,000 cells in 50 μL of phosphate buffered saline solution (16×10⁶ cells per mL). Function (resting anal sphincter pressures) and histology were evaluated 8 weeks after treatment (n=8 per group). A separate group of rats (n=6 per group outlined below) was used to investigate cytokine expression 7 days after treatment.

### Sample Size Estimation

Based on that our previous work with a 0.8 power and adjusted significance level of 0.5 with a conservative Tukey-Kramer method for multiple comparisons, a sample size of 8 animals per group (3 treatments and 3 time points) was calculated for functional outcomes.

### Anal Manometry

Resting anal pressure (RP) was assessed before excision, as well as before and 8 weeks after treatment, as per our previous protocol. Sun et al., Dis Colon Rectum, 59, 434-442 (2016). Under anesthesia, a 7-F T-Doc air-charged catheter (Laborie Medical Technologies, Mississauga, Ontario, Canada) was inserted into the anal canal and connected to a Goby Anorectal Manometry System (Laborie Medical Technologies) for RP recording. Eight typical RP waves (a stable baseline pressure) were collected for analysis at each of the 3 investigational time points. The average of the 8 measured RPs in each animal was used for additional group analysis.

### Histology

Anal sphincter histology was evaluated 8 weeks after treatment. Masson trichrome staining was performed as described previously. Sun et al., ibid. After euthanasia, the anal tissues were harvested and fixed with 10% formalin before being paraffin embedded for histology. For each specimen, Masson trichrome staining was performed on serial transverse sections (5 μm thick, 50 μm apart) along the 1.5 mm-long anal canal tissue starting at the anal verge. The sections were viewed and scanned by an observer blinded to group assignments under a bright-field microscope at ×20 magnification using the Leica SCN400 Slide Scanner (Leica Microsystems Inc, Buffalo Grove, Ill.). The site of injury was recognized by the disruption of the anal sphincter complex in cross-section.

Quantification of muscle and connective tissue was performed using Image-Pro Plus 7.0 software (Media Cybernetics, Rockville, Md.). Volumetric analysis of each section was done as described previously. Sun et al., Dis Colon Rectum, 60:416-425 (2017) Briefly, in the same histological section, we assessed the percentage of muscle (circular red fiber-like bundles, usually disorganized, in the area of the created defect on Masson staining) and connective tissue (collagen-rich fibrosis). The proportion of muscle in the region of the defect was calculated as muscle area divided by the area of muscle plus the area of connective tissue in the region of the defect. The proportion of connective tissue in the region of the defect was calculated as connective tissue area divided by the area of muscle plus the area of connective tissue in the region of the defect. The proportion of muscle in the intact region was calculated as muscle area divided by the area of muscle plus the area of connective tissue in the intact region. The proportion of connective tissue in the intact region was calculated as connective tissue area divided by the area of muscle plus the area of connective tissue in the intact region. Results are reported as the ratio of muscle and connective tissue at the defect to that in the intact area.

### Immunohistochemistry

The paraffin-embedded, formalin-fixed rat anal canal samples were sectioned at 5 μm. Immunohistochemistry staining was performed using a Discovery ULTRA automated stainer (Ventana Medical System Inc, Tucson, Ariz.). In brief, antigen retrieval was performed using a tris/borate/EDTA buffer (Discovery CC1, Ventana Medical Systems, Inc, Oro Valley, Ariz.)(pH 8.0 to 8.5) for 64 minutes at 95° C. Slides were incubated with Desmin antibody-1 (D33) at a 1:40 dilution (MS-376-S; Thermo Scientific, Fremont, Calif.) for 1 hour at room temperature. The primary antibody was visualized using the OmniMap antimouse horseradish peroxidase secondary antibody (Ventana Medical Systems) and the ChromoMap DAB detection kit (Ventana Medical Systems). Lastly, the slides were counterstained with hematoxylin and eosin.

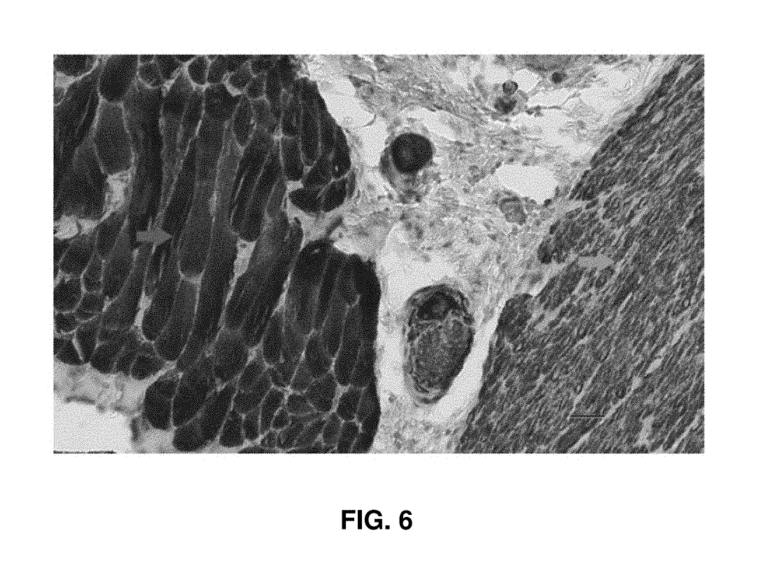

The smooth muscle internal anal sphincter (IAS) and striated muscle external anal sphincter (EAS) were identified based on analysis of Desmin-stained sections (FIG. 6). The EAS was identifiable as dark-brown stained tissue with striations, whereas the IAS was identifiable as light-brown staining without the striated structure. Volumetric analysis was performed as described previously. Sun et al., Dis Colon Rectum, 60:416-425 (2017) Sections were viewed and scanned under a bright-field microscope at ×20 magnification using the Leica SCN400 Slide Scanner. Quantification of each muscle was performed using Image-Pro Plus 7.0 software. We assessed the volume of both IAS and EAS muscles at the area of defect and in the intact area separately and evaluated the volume as a percentage of the total muscle at the site of the defect and the intact area. We then compared the individual muscles as a ratio of that percentage at the defect with that in the intact area.

### Cytokine Expression

Seven days posttreatment, anal canal tissue of 5 mm length was harvested before it was preserved in liquid nitrogen. Tissue was prepared using a lysate solution in a 1:10 ratio of tissue weight:lysate volume (which includes 10% cell lysis buffer No. 9803, Cell Signaling Technology, Danvers, Mass.; and 1% (milligrams per milliliter) protease inhibitor tablet No. 8820, Sigma-Aldrich, St. Louis, Mo.). Western blots were performed using primary antibodies to CXCR4 (No. ab124824, Abcam, Cambridge, Mass.) and Myf5 (No. ab125078, Abcam), with anti-β-actin (No. sc47778, Santa Cruz Biotechnology, Santa Cruz, Calif.) as the endogenous control. Fluorescence dye-labeled secondary antibodies (LICOR, Lincoln, Nebr.) were mixed with IRDye 800CW Donkey antirabbit (No. 926-32213, green) for both CXCR4 and Myf5 and IRDye 680RD Donkey antimouse for β-actin (No. 926-68072, red). An Odyssey CLx infrared imaging system (LI-COR) was used for band imaging and quantification of cytokine expression. The ratio of CXCR4 and Myf5 to endogenous β-actin of each sample was calculated, and the final result is shown normalized to the mean of the injury alone group (n=6).

### Statistical Analysis

Parametric group comparisons for anal manometry over time with respect to resting pressure and measures of change were performed using ANOVA with pairwise group comparisons using a t test with a Bonferroni correction such that p<0.0083 was regarded as significant. Quantification for histology, immunohistochemistry, and cytokine expression was performed using a 1-way ANOVA followed by a Tukey test in SigmaPlot 11.0 (Systat Software Inc, San Jose, Calif.), with p<0.05 indicating a statistically significant difference in all comparisons. Results are presented as mean±SD of data from 6 (Western blot) or 8 (manometry and histology) animals per group.

### Results

### MSC Identification

The multipotent features of the MSC was confirmed by their differentiation capability into adipogenic, chondrogenic, and osteogenic cells ex vivo.

### Anal Manometry

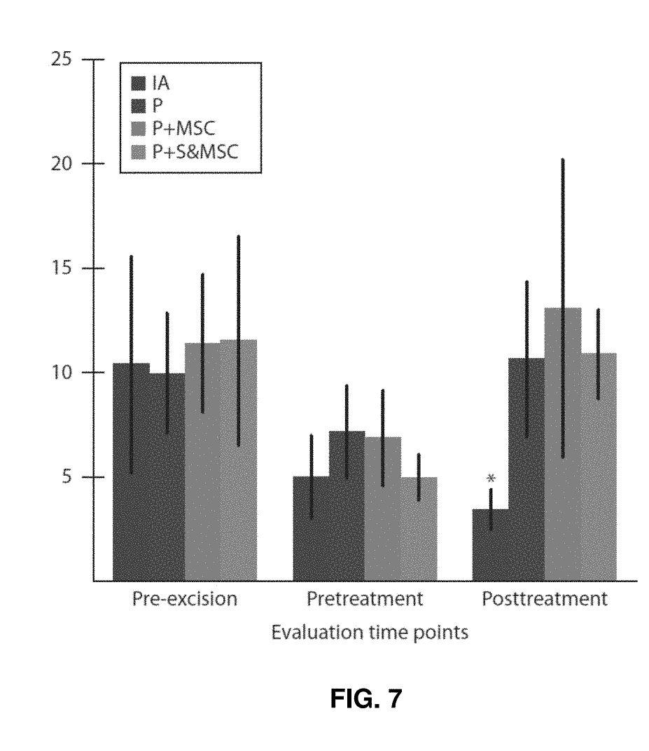

Before the injury, there was no significant difference in resting pressure among the 4 groups (IA, 10.4±5.08 cm H₂O; P, 10.0±2.85 cm H₂O; P+MSC, 11.4±3.27 cm H₂O; P+S&MSC, 11.5±4.97 cm H₂O; p>0.0083). Three weeks after injury and before treatment, no significant difference was noted between the groups (IA, 5.0±1.98 cm H₂O; P, 7.2±2.20 cm H₂O; P+MSC, 6.9±2.24 cm H₂O; P+S&MSC, 5.0±1.06 cm H₂O). Eight weeks after treatment, all of the SDF-1 plasmid-treated groups had significantly higher pressures than the IA group (IA, 3.4±0.96 cm H₂O; P, 10.6±3.70 cm H₂O, p=0.001; P+MSC, 13.1±7.07 cm H₂O, p<0.001; P+S&MSC, 10.9±2.11 cm H₂O, p<0.001; FIG. 7). No significant differences in anal pressure were noted between animals receiving the SDF-1 plasmid and those receiving the plasmid and cells or scaffold.

Anal pressure increase from pretreatment to post-injury was significantly increased in all the SDF-1 plasmid-treated groups compared with the IA group, which showed a significant decrease in pressure (IA, −1.6±1.49 cm H₂O; P, 3.5±3.39 cm H₂O, p=0.004; P+MSC, 6.2±5.94 cm H₂O, p=0.007; P+S&MSC, 5.9±2.97 cm H₂O, p<0.001). No significant difference was noted among the 3 SDF-1 plasmid-treated groups in the change in pressure after-treatment (p>0.0083).

### Histology and Immunohistochemistry

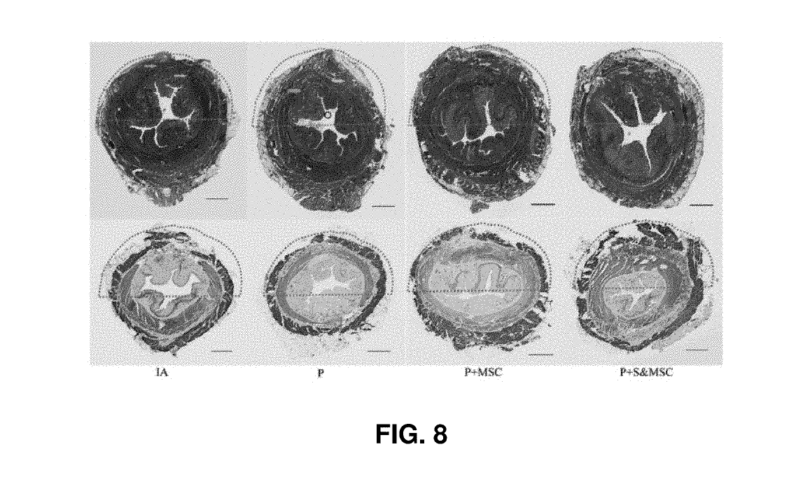

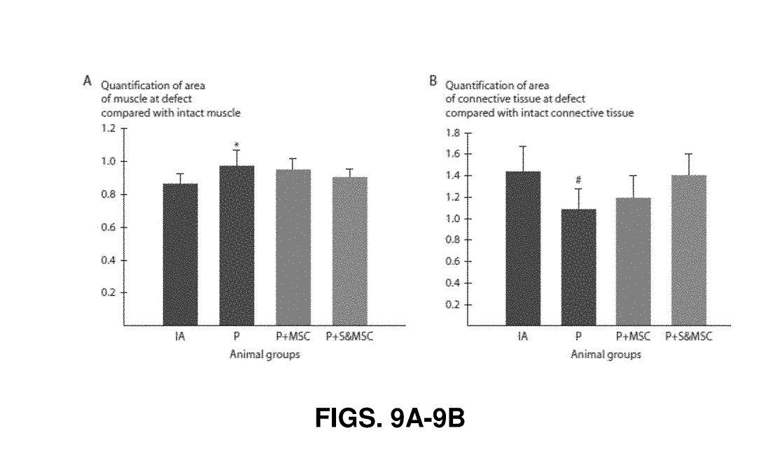

All three of the SDF-1 plasmid-treated groups showed filling of the defect with muscle fibers, whereas the area of the defect in the IA group showed disorganized architecture with patchy filling of the defect (FIG. 8). Quantification of the total muscle at the site of injury revealed that, compared with the IA group, the plasmid alone group had significantly more muscle (IA, 0.86±0.06; P, 0.97±0.09; p=0.03). No significant difference in total muscle quantification was noted on the other intergroup comparisons (P+MSC, 0.95±0.07; P+S&MSC, 0.90±0.05; FIG. 9A).

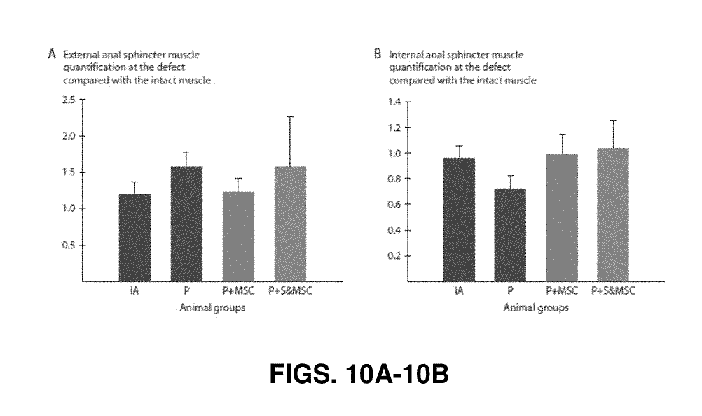

Significantly less fibrosis was seen in the SDF-1 plasmid alone group compared with the IA and P+S&MSC groups (P, 0.97±0.09; IA, 1.44±0.24, p=0.018; P+S&MSC, 1.40±0.21, p=0.03). No significant difference was noted between the other SDF-1 plasmid-treated groups on intergroup comparisons (P+MSC, 1.19±0.22; FIG. 9B). No significant differences were found between the groups in the proportion of muscle in the defect region and intact areas of the IAS muscle (IA, 0.95±0.27; P, 0.72±0.3; P+MSC, 0.99±0.45; P+S&MSC, 1.03±0.64; p=0.52; FIG. 10A) or the EAS muscle (IA, 1.20±0.47; P, 1.58±0.56; P+MSC, 1.24±0.49; P+S&MSC, 1.57±1.98; p=0.85; FIG. 10B).

### Cytokine Expression

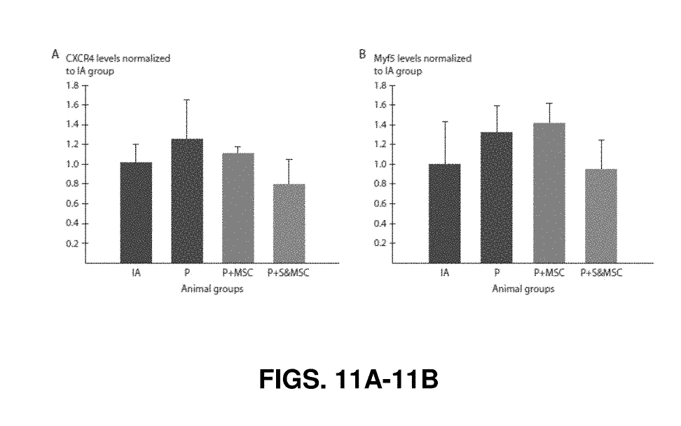

Seven days after treatment, there were no significant differences between the groups in CXCR4 (IA, 1.20±0.47; P, 1.58±0.56; P+MSC, 1.24±0.49; P+S&MSC, 1.57±1.99; p=0.37) or Myf5 (IA, 1.00±0.43; P, 1.32±0.27; P+MSC, 1.42±0.20; P+S&MSC, 0.98±0.3; FIGS. 11A & 11B).

### Discussion

The quest to regenerate muscles in the region of the anal sphincter is ongoing. Early studies evaluated the concept of anal sphincter regeneration after an acute injury, and most recent studies are still centered on this concept. Mazzanti et al., Stem Cell Res Ther., 7:85 (2016) Clinical trials using stem cells also use the cells after a repair of the anal sphincter and inject into the cut ends. Sarveazad et al., Stem Cell Res Ther., 8:40 (2017). However, the challenge is in regenerating functional muscle at a time when the tissue environment for regeneration is quiescent long after injury.

To change the tissue environment, few options have been studied. We have researched electrical stimulation as a low-grade injury and have reported on retention of exogenous MSCs and regeneration of the muscles. Sun et al., Dis Colon Rectum., 59:434-442 (2016) One clinical trial has used electrical stimulation for 21 days, followed by injection of cells and reported significant increases in anal pressures and quality-of-life scores with a decrease in incontinence episodes, incontinence scores, and frequency of bowel movements 5 years after treatment. Frudinger et al., Gut., 59:55-61 (2010) Other therapies that have been evaluated are shock wave therapy (Romeo et al., Med Princ Pract., 23:7-13 (2014)) and platelet-rich plasma. Chung et al., Am J Sports Med., 41:2909-2918 (2013).

Advances in cellular therapy have not focused on muscle regeneration in the absence of inflammation. Apart from the studies on the anal sphincter, studies involving regeneration of volumetric muscle loss or tendon injuries have shown slow progress because of regulatory issues, poor donor cell viability, and engraftment issues. Sicari et al., Anat Rec (Hoboken). 297:51-64 (2014). To regenerate an anal sphincter that is deficient in its continuity, the treatment needs not only to fill a defect with muscle but that which is functional.

A functionally effective anal sphincter depends on both the IAS and EAS complexes being intact and innervated. The large muscle defect that we created disrupted the anal sphincter complex, requiring regeneration and reinnervation of the smooth and striated muscles of the anal sphincters. Previous work has also demonstrated recovery of the neuromuscular system of the anal sphincter in acute (Brügger et al., Int J Colorectal Dis., 29:1385-1392 (2014)), as well as a chronic, injury models. Sun et al., Dis Colon Rectum. 60:416-425 (0.2017). Hence, there is a need to focus on regeneration of viable muscle with innervation to achieve continence control.

Bitar et al. have investigated the possibility of regenerating the IAS from GI cells. Bitar et al., Gastroenterology, 146:1614-1624 (2014). Their emphasis is to replace the IAS with an engineered gut sphincter complex, composed of human smooth muscle and neural progenitor cells and engineered on a scaffold, which has been successfully implanted in rodents. They have demonstrated both innervation and ex vivo muscle tensile strength of the regenerated composite. Zakhem et al., J Tissue Eng Regen Med., 11(12):3398-3407 (2017). Kajbafzadeh et al37 have also implanted myogenic cells on a decellularized EAS in a rabbit model. Kajbafzadeh et al., Ann Biomed Eng., 44:1773-1784 (2016) Their results show that a decellularized EAS implanted with myogenic cells from a thigh muscle can improve anal sphincter contractility in the short term, whereas in the long term, 2 years later, both seeded and nonseeded decellularized anal sphincter matrices had equivalent results. Although the authors state that this may be an option for treating fecal incontinence, their model is that of a complete external sphincter excision, and it is unclear whether they are suggesting that the EAS can be augmented without excising it. Clinically, translation of both of these lines of research would fit a clinical indication similar to that of an artificial anal sphincter.

In our previous study we evaluated the same groups at an earlier time point of 4 weeks. We demonstrated an increase in anal sphincter pressures in the group treated with the SDF-1 plasmid alone and the group that received the plasmid along with the exogenous MSCs. Histology was significant for a greater reorganization of muscle, and muscle when quantified was increased in the plasmid plus MSC group compared with the injury alone group.

The current study has shown that, at a time when the process of innate regeneration from an injury has waned, a plasmid encoding SDF-1 results in regenerated muscle to bridge an entire hemicircumference and not just a small defect. These effects have been improved since our last study, which evaluated results 4 weeks after treatment and have increased the muscle volume in the same proportion as that seen in the uninjured area. In addition, for the first time, a therapy has regenerated both smooth and skeletal muscles after an injury. Not only have both muscles regenerated, but it is in the same ratio as that present in the uninjured area, suggesting that, although the plasmid may be expressed for <30 days, there seem to be factors that modulate this effect and may prevent excessive muscle regeneration. However, a longer-term study in rodents should also be performed to evaluate any additional growth of muscle beyond 8 weeks.

Anal sphincter pressure was also increased to near normal values and sustained over 8 weeks. This is one of the criterion required to translate this research, because muscle without tone would be redundant. However, we did not examine the tensile strength ex vivo of the regenerated muscle fibers. Another limitation is that, because we did not block the action of SDF-1, we could not demonstrate that the reported effects were from SDF-1 alone. Future research should also examine muscle innervation and angiogenesis of the regenerated muscle.

CXCR4 is a receptor for the ligand CXCL12 (SDF-1) and has been involved in chemotaxis of leukocytes in specific inflammatory conditions. Debnath et al., Theranostics, 3:47-75 (2013) The CXCL12/CXCR4 chemokine axis is involved in the recruitment of stem cells from bone marrow and other tissues and signaling involved in chemotaxis, cell survival, proliferation, increase in intracellular calcium, and gene transcription. Teicher B A, Fricker S P., Clin Cancer Res., 16:2927-2931 (2010) We have shown previously that there was an increase in satellite cells after plasmid therapy and have inferred that this may be the cause of muscle regeneration. MyF5 is one of the factors responsible for myogenic specification and differentiation. Tapscott S J., Development, 132:2685-2695 (2005). However, both of these cytokines were not differentially expressed in this study. Hence, we have not conclusively ascertained the molecular mechanisms that could cause this effect. Large animal studies will focus on this aspect.

### Conclusion

In a rat model of a large anal sphincter injury, the plasmid encoding for SDF-1 regenerated both smooth and skeletal muscles. Increased muscle regeneration and increased anal sphincter pressures were sustained over 8 weeks. The addition of MSCs with or without a scaffold did not enhance this effect. Future research should be directed toward detecting muscle tensile strength of the regenerated muscle and the molecular mechanisms that could cause and inhibit it.

### Example 3: SDF-1 Plasmid to Regenerate the Anal Sphincter in a Pig Model

Regeneration of a chronic anal sphincter defect is a challenge. Most preclinical studies have evaluated regeneration after an acute injury or after an injury and a repair. Pathi et al., Obstetrics and gynecology, 119(1):134-44 (2012). All have reported various degrees of regeneration, changes in anal resting pressures, tensile strength and muscle fatigue as outcomes. The challenge is in replicating this process in an area that is quiescent and has no ongoing processes that involve any innate repair mechanisms.

Oh et al. have used polycaprolaptone beads in a dog model which was loaded with basic fibroblastic growth factor and autologous myoblasts. Oh et al., Dis Colon Rectum, 58(5):517-25 (2015). Their model was an excision of 25% of the anal sphincter muscle. Their hypothesis was that the fibroblastic growth factor prolonged effect of the stem cells which affected regeneration. In prior studies we have used a cytokine called stromal cell derived factor-1 (SDF-1) in a rodent model of a chronic anal sphincter defect. SDF-1 is a cytokine that has been shown to affect tissue regeneration by stimulating neo-angiogenesis and attracts bone marrow derived stem cells to the area where it is injected. Zhang et al., Faseb J. 21(12): 3197-207 (2007).

We have previously established that in muscle tissue SDF-1 appears to increase the number of satellite cells in the muscles where it is injected and thereby affects tissue regeneration. We have shown that excision of 50% of the circumference of the anal sphincter and treatment 3 weeks with SDF 1 alone or in conjunction with stem cells or also in a scaffold with stem cells later resulted in increased anal sphincter pressures, regenerated internal and external anal sphincter muscle while the control animals had low sphincter pressures with a disorganized repair area. Sun et al., Dis Colon Rectum, 60(4):416-25 (2017).

In this example we have used a larger animal model to study the effects treatment with SDF 1 injection into the area of the repair 4 weeks after an injury. We have used 2 different doses to establish if a second dose creates a greater response to regeneration. Our hypothesis is that in a minipig model of a chronic large anal sphincter defect SDF-1 improves resting anal tone and results in regeneration of the defect with functional muscle tissue.

### Material and Methods

### SDF-1-Encoded Non-viral Plasmid

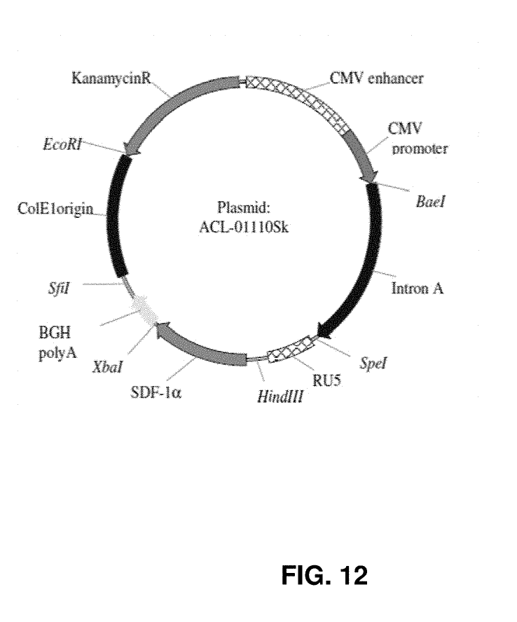

The SDF-1-encoded plasmid was obtained from Juventas Therapeutics Inc. (Cleveland, Ohio), and was the same product that was used in the previous study. Aghaee-Afshar et al., Dis Colon Rectum, 52(10):1753-61 (2009). An image of the plasmid is shown in FIG. 12. This study used the SDF-1 plasmid in dextrose solution with a concentration of 2 mg/ml.

### Mini Pig Model of a Chronic Large Anal Sphincter Defect

This study used age and weight-matched Yorkshire mini pig (25-30 kg). To create an anal sphincter defect, the miniature pig (mini-pig) was anesthetized with an intramuscular injection of ketamine (20 mg/kg) and xylazine (2 mg/kg) followed by tracheal intubation with isoflurane in oxygen (1-3%). A peripheral catheter was be placed in an auricular vein for administration of fluids. The surgical site was shaved and cleaned with Betadine solution. After anesthesia the animals underwent an anal manometer and anal ultrasound. All the animals were then subjected to an incision which was made just below the anal verge on the posterior aspect. The skin was dissected from the underlying tissue. The muscle was identified and pick up in a babcock forceps. The muscle was dissected to the ends of the incision. A Bovie was used to excise the muscle such that a defect of 180 degrees was created. Hemostasis was secured using the Bovie and the skin was closed using interrupted sutures. One cc of Marcaine 0.25% was injected under the incision for pain control. The animals were allowed to recover for 6 weeks.

Six weeks after surgery, the animals were subjected to a manometry and anal ultrasound testing. After completion of these tests the pigs were randomly allocated to receive an injection of either SDF-1 (1 mg/1 ml) or saline (1 ml) on the two cut ends of the muscle at the edge of the defect. The pigs were divided into three groups based on the treatment they received: saline group (n=5), 1-injection SDF-1 group (n=9), and 2-injection SDF-1 group (n=5). The animals in the one injection group received a single injection 6 weeks after injury. While those in the 2 injection group received SDF-1 injection at 6-week and 8-week after the surgery. Eight weeks after the last SDF-1 injection, the animals underwent a repeat manometry and an anal ultrasound before they were euthanized. The anal canal tissue was harvested and fixed. The site of injury was recognized by a suture placed in the middle of posterior circumference of anal canal.

### Anal Manometry

Anal manometry was carried out as per on our previous published protocol using an adult catheter. Sun et al., Dis Colon Rectum, 60(12):1320-8 (2017). In brief, under anesthesia, a 19Fr T-Doc air-charged anorectal manometry catheter (Laborie Medical Technologies, Mississauga, Canada) was inserted into the anal canal. The catheter was connected to Goby Anorectal Manometry System (Laborie Medical Technologies, Mississauga, Canada) for data collection of eight typical resting pressure (RP) waves (a stable baseline pressure) at the posterior site as well as the average of posterior, anterior, left and right sites. The pressure data was collected at three time points: just before creation of the defect (pre-injury), before the first SDF-1 injection (pre-treatment), and 8 weeks after the last SDF-1 injection (post-treatment). The average data of 8 resting pressures (RP) per animal were used for analysis.

### Histology Analysis

After the animals were euthanized, anal canal histology was done at post-treatment. The anal tissues were harvested and fixed with 10% formalin solution (SIGMA-ALDRICH, Cleveland, Ohio) before being embedded in paraffin. The anal canal specimens were processed as serial sections (5 μm thick, 300-500 μm apart) starting at a distance of 3-3.5 mm from the anal edge.

### Muscle Regeneration Quantification

Masson's Trichrome stained sections were scanned and read by a blinded observer under LEICA DMI600 B inverted microscope and LASX program (Leica Microsystems Inc., Buffalo Grove, Ill.). Quantification analysis of muscle and connective tissue (CT) was processed using Image-Pro Plus 7.0 software (Media Cybernetics, Rockville, Md.). Firstly the muscular (circular red fiber-like bundles, usually disorganized, in the area of the defect on Masson's staining) and fibrosis (blue collagen-rich tissue) were identified. Then the defect half (posterior of anal cross-section was divided into 3 even parts, the ratio of muscle to connective tissue were analyzed at the site of side (which is the average of 2⅓ parts next to the end of the defect) and the site of the middle ⅓ part. The ratio was compared among animal groups.

### Immunohistochemistry for Desmin Detection

Immunohistochemistry (IHC) staining of Desmin was performed using a revised version of our published protocol. Aghaee-Afshar et al., Dis Colon Rectum, 52(10):1753-61 (2009) In brief, slides were incubated with Desmin antibody-1 (D33) at a 1:200 dilution (MS-376-S; Thermo Scientific, Fremont, Calif.) for 1 hour at room temperature. The primary antibody was visualized using the OmniMap anti-mouse horseradish peroxidase secondary antibody (Ventana Medical Systems) and the ChromoMap DAB detection kit (Ventana Medical Systems). Lastly, the slides were counterstained with hematoxylin and eosin. The striated muscle external anal sphincter (EAS) (brown stained tissue with striation) and the smooth muscle IAS (light-brown staining without the striation structure) were identified based on analysis of Desmin-stained sections.

### Statistical Analysis

SigmaPlot 11.0 (Systat Software Inc, San Jose, Calif.) was used and the results were expressed as mean±standard deviation (SD). One way ANOVA followed by Tukey test was used to analyze the manometry data and histological quantification analysis, p<0.05 was regarded as a statistically significant difference in all comparisons.

### Results

### Manometry Result

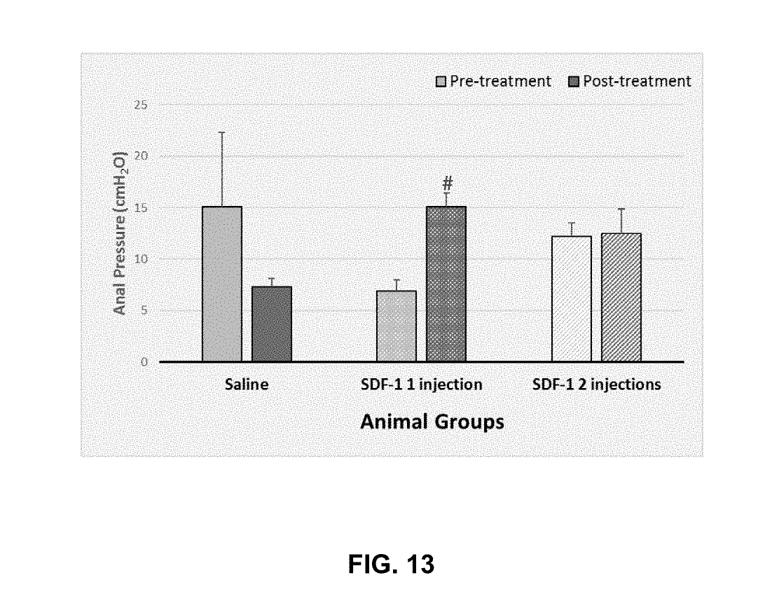

Analysis of pressure recorded at the posterior channel of the manometry catheter is shown in FIG. 13. At the pretreatment timepoint no significant difference was found among 3 groups in the mean RP. After treatment both SDF-1 1 injection (1-SDF-1) (p=0.003) and SDF-1 2 injections (2-SDF-1) (p=0.004) groups had significantly higher mean pressure compared to the saline group. No significant difference was found between the two SDF-1 injection groups after treatment. When comparing the different time points within each group: there was no difference in the saline group, however significant difference was found among 3 time points in the SDF-1 treated groups. In 1-SDF-1 group, at the post-treatment time point the pressure was significantly higher pressure than at both pre-injury (p<0.001) and pre-treatment time points (p<0.001). In the 2-SDF-1 group, post-treatment had significantly higher pressure than pre-injury (p=0.036).

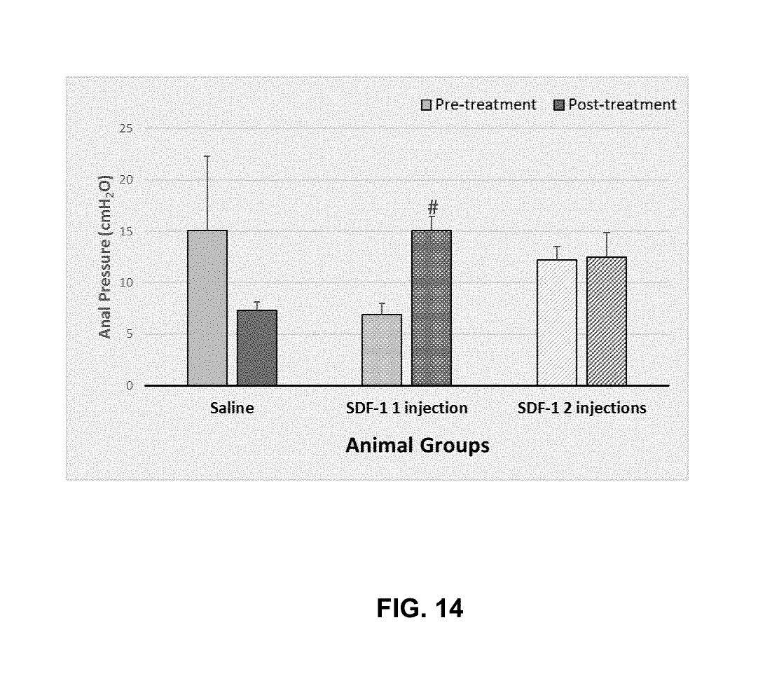

Average pressure analysis is shown in FIG. 14. At pre-injury or post-treatment, no significant difference was found among three groups. When comparing the anal RP between 3 time points within animal groups, there was no significant difference was found in saline group; in 1-SDF-1 group, the post-treatment RP had significantly higher pressure than both pre-injury (p<0.001) and pre-treatment time points (p<0.001); no significant difference was found within 2-SDF-1 group.

### Discussion

There are very few studies that have dealt with the issue of a chronic anal sphincter injury. This study examines a chronic injury in a large animal model. Anal sphincters have been described by removal of the internal anal sphincter muscle or the external anal sphincter muscle. Raghavan et al. have evaluated a construct that is created by using gastrointestinal smooth muscle cells from a tissue biopsy and grown in vitro and reimplanted into the area of an internal anal sphincter muscle. Raghavan et al., Gastroenterology, 141(1):310-9 (2011). They have demonstrated angiogenesis, development of neuro filaments and functional muscle tissue. Another study has demonstrated a 3-D construct of an external anal sphincter muscle in a rabbit model. Kajbafzadeh et al., Ann Biomed Eng., 44(5):1773-84 (2016). These applications are different then treating a defect in the anal sphincter muscle-suited to the same indications as those of an artificial anal sphincter. The only 2 studies that have used chronic anal sphincter defects were one in a human trial where these pre-stimulated the anal sphincter using and electrode for 21 days before a sphincter repair and injection of stem cells and continued this treatment after surgery. They reported outcomes at one year and 5 years with increase in incontinence scores although they did not show marked increase in the anal sphincter tone. Frudinger et al., Colorectal Dis., 17(9):794-801 (2015). In the dog model of a chronic anal sphincter injury with the polycaprolaptone beads Oh et al. (ibid) have created a chronic anal sphincter injury and injected the beads. They did not have an arm with only the beads with or without the cytokine fibroblastic growth factor in it. Therefore it was not clear whether the actions of the regenerated muscle but due to the cytokine or the stem cells or a bulking effect.

This example has demonstrated increase in resting pressures after a large injury and 1 and 2 injections of the SDF-1 plasmid. Although detailed histology is still awaited, qualitative description of regeneration is more pronounced in the SDF-1 group.

This study corroborates the study in the rodent model. The pig anal sphincter is slightly smaller than human anal sphincter complex and translation should take that into consideration.

### CONCLUSION

Eight weeks after a single dose of SDF-1 injected 6 weeks after an excision of 50% of the circumference of the anal sphincter improved resting anal sphincter pressures, and regenerated muscle in the entire area of the defect. SDF-1 plasmid is safe and effective in treating chronic defects of the anal sphincter in a large animal and can be translated.

SDF-1 sequences
SEQ ID NO: 1:
KPVSLLYRCPCRFFESHVARANVKHLKILNTPNCALQIVARLKNNNRQVCIDPKLKWIQ EYLEKALNK
SEQ ID NO: 2:
MNAKVVVVLVLVLTALCLSDGKPVSLSYRCPCRFFESHVARANVKHLKILNTPNCALQIVARLKNNNRQ
VCIDPKLKWIQEYLEKALNK
SEQ ID NO: 3:
MDAKVVAVLALVLAALCISDGKPVSLSYRCPCRFFESHVARANVKHLKILNTPNCALQIVARLKSNNRQ
VCIDPKLKWIQEYLDKALNK
SEQ ID NO: 4:
GCCGCACTTTCACTCTCCGTCAGCCGCATTGCCCGCTCGGCGTCCGGCCCCCGACCCGCGCTCGTCCGC
CCGCCCGCCCGCCCGCCCGCGCCATGAACGCCAAGGTCGTGGTCGTGCTGGTCCTCGTGCTGACCGCGC
TCTGCCTCAGCGACGGGAAGCCCGTCAGCCTGAGCTACAGATGCCCATGCCGATTCTTCGAAAGCCATG
TTGCCAGAGCCAACGTCAAGCATCTCAAAATTCTCAACACTCCAAACTGTGCCCTTCAGATTGTAGCCC
GGCTGAAGAACAACAACAGACAAGTGTGCATTGACCCGAAGCTAAAGTGGATTCAGGAGTACCTGGAGA
AAGCTTTAAACAAGTAAGCACAACAGCCAAAAAGGACTTTCCGCTAGACCCACTCGAGGAAAACTAAAA
CCTTGTGAGAGATGAAAGGGCAAAGACGTGGGGGAGGGGGCCTTAACCATGAGGACCAGGTGTGTGTGT
GGGGTGGGCACATTGATCTGGGATCGGGCCTGAGGTTTGCCAGCATTTAGACCCTGCATTTATAGCATA
CGGTATGATATTGCAGCTTATATTCATCCATGCCCTGTACCTGTGCACGTTGGAACTTTTATTACTGGG
GTTTTTCTAAGAAAGAAATTGTATTATCAACAGCATTTTCAAGCAGTTAGTTCCTTCATGATCATCACA
ATCATCATCATTCTCATTCTCATTTTTTAAATCAACGAGTACTTCAAGATCTGAATTTGGCTTGTTTGG
AGCATCTCCTCTGCTCCCCTGGGGAGTCTGGGCACAGTCAGGTGGTGGCTTAACAGGGAGCTGGAAAAA
GTGTCCTTTCTTCAGACACTGAGGCTCCCGCAGCAGCGCCCCTCCCAAGAGGAAGGCCTCTGTGGCACT
CAGATACCGACTGGGGCTGGGCGCCGCCACTGCCTTCACCTCCTCTTTCAACCTCAGTGATTGGCTCTG
TGGGCTCCATGTAGAAGCCACTATTACTGGGACTGTGCTCAGAGACCCCTCTCCCAGCTATTCCTACTC
TCTCCCCGACTCCGAGAGCATGCTTAATCTTGCTTCTGCTTCTCATTTCTGTAGCCTGATCAGCGCCGC
ACCAGCCGGGAAGAGGGTGATTGCTGGGGCTCGTGCCCTGCATCCCTCTCCTCCCAGGGCCTGCCCCAC
AGCTCGGGCCCTCTGTGAGATCCGTCTTTGGCCTCCTCCAGAATGGAGCTGGCCCTCTCCTGGGGATGT
GTAATGGTCCCCCTGCTTACCCGCAAAAGACAAGTCTTTACAGAATCAAATGCAATTTTAAATCTGAGA
GCTCGCTTTGAGTGACTGGGTTTTGTGATTGCCTCTGAAGCCTATGTATGCCATGGAGGCACTAACAAA
CTCTGAGGTTTCCGAAATCAGAAGCGAAAAAATCAGTGAATAAACCATCATCTTGCCACTACCCCCTCC
TGAAGCCACAGCAGGGTTTCAGGTTCCAATCAGAACTGTTGGCAAGGTGACATTTCCATGCATAAATGC
GATCCACAGAAGGTCCTGGTGGTATTTGTAACTTTTTGCAAGGCATTTTTTTATATATATTTTTGTGCA
CATTTTTTTTTACGTTTCTTTAGAAAACAAATGTATTTCAAAATATATTTATAGTCGAACAATTCATAT
ATTTGAAGTGGAGCCATATGAATGTCAGTAGTTTATACTTCTCTATTATCTCAAACTACTGGCAATTTG
TAAAGAAATATATATGATATATAAATGTGATTGCAGCTTTTCAATGTTAGCCACAGTGTATTTTTTCAC
TTGTACTAAAATTGTATCAAATGTGACATTATATGCACTAGCAATAAAATGCTAATTGTTTCATGGTAT
AAACGTCCTACTGTATGTGGGAATTTATTTACCTGAAATAAAATTCATTAGTTGTTAGTGATGGAGCTT
AAAAAAAA
SEQ ID NO: 5:
CATGGACGCCAAGGTCGTCGCTGTGCTGGCCCTGGTGCTGGCCGCGCTCTGCATCAGTGACGGTAAGCC
AGTCAGCCTGAGCTACAGATGCCCCTGCCGATTCTTTGAGAGCCATGTCGCCAGAGCCAACGTCAAACA
TCTGAAAATCCTCAACACTCCAAACTGTGCCCTTCAGATTGTTGCAAGGCTGAAAAGCAACAACAGACA
AGTGTGCATTGACCCGAAATTAAAGTGGATCCAAGAGTACCTGGACAAAGCCTTAAACAAGTAAGCACA
ACAGCCCAAAGGACTT
SEQ ID NO: 6:
AGATCTCCTAGGGAGTCCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGACGTCAA
TGGGTGGAGTATTTACGGTAAACTGCCCACTTGGCAGTACATCAAGTGTATCATATGCCAAGTACGCCC
CCTATTGACGTCAATGACGGTAAATGGCCCGCCTGGCATTATGCCCAGTACATGACCTTATGGGACTTT
CCTACTTGGCAGTACATCTACGTATTAGTCATCGCTATTACCATGGTGATGCGGTTTTGGCAGTACATC
AATGGGCGTGGATAGCGGTTTGACTCACGGGGATTTCCAAGTCTCCACCCCATTGACGTCAATGGGAGT
TTGTTTTGGCACCAAAATCAACGGGACTTTCCAAAATGTCGTAACAACTCCGCCCCATTGACGCAAATG
GGCGGTAGGCGTGTACGGTGGGAGGTCTATATAAGCAGAGCTCGTTTAGTGAACCGTCAGATCGCCTGG
AGACGCCATCCACGCTGTTTTGACCTCCATAGAAGACACCGGGACCGATCCAGCCTCCGCGGCCGGGAA
CGGTGCATTGGAACGCGGATTCCCCGTGCCAAGAGTGACGTAAGTACCGCCTATAGAGTCTATAGGCCC
ACCCCCTTGGCTTCTTATGCATGCTATACTGTTTTTGGCTTGGGGTCTATACACCCCCGCTTCCTCATG
TTATAGGTGATGGTATAGCTTAGCCTATAGGTGTGGGTTATTGACCATTATTGACCACTCCCCTATTGG
TGACGATACTTTCCATTACTAATCCATAACATGGCTCTTTGCCACAACTCTCTTTATTGGCTATATGCC
AATACACTGTCCTTCAGAGACTGACACGGACTCTGTATTTTTACAGGATGGGGTCTCATTTATTATTTA
CAAATTCACATATACAACACCACCGTCCCCAGTGCCCGCAGTTTTTATTAAACATAACGTGGGATCTCC
ACGCGAATCTCGGGTACGTGTTCCGGACATGGGCTCTTCTCCGGTAGCGGCGGAGCTTCTACATCCGAG
CCCTGCTCCCATGCCTCCAGCGACTCATGGTCGCTCGGCAGCTCCTTGCTCCTAACAGTGGAGGCCAGA
CTTAGGCACAGCACGATGCCCACCACCACCAGTGTGCCGCACAAGGCCGTGGCGGTAGGGTATGTGTCT
GAAAATGAGCTCGGGGAGCGGGCTTGCACCGCTGACGCATTTGGAAGACTTAAGGCAGCGGCAGAAGAA
GATGCAGGCAGCTGAGTTGTTGTGTTCTGATAAGAGTCAGAGGTAACTCCCGTTGCGGTGCTGTTAACG
GTGGAGGGCAGTGTAGTCTGAGCAGTACTCGTTGCTGCCGCGCGCGCCACCAGACATAATAGCTGACAG
ACTAACAGACTGTTCCTTTCCATGGGTCTTTTCTGCAGTCACCGTCCTTGCCATCGGTGACCACTAGTG
GCTCGCATCTCTCCTTCACGCGCCCGCCGCCCTACCTGAGGCCGCCATCCACGCCGGTTGAGTCGCGTT
CTGCCGCCTCCCGCCTGTGGTGCCTCCTGAACTGCGTCCGCCGTCTAGGTAAGTTTAAAGCTCAGGTCG
AGACCGGGCCTTTGTCCGGCGCTCCCTTGGAGCCTACCTAGACTCAGCCGGCTCTCCACGCTTTGCCTG
ACCCTGCTTGCTCAACTCTACGTCTTTGTTTCGTTTTCTGTTCTGCGCCGTTACAGATCGGTACCAAGC
TTGCCACCACCATGAACGCCAAGGTCGTGGTCGTGCTGGTCCTCGTGCTGACCGCGCTCTGCCTCAGCG
ACGGGAAGCCCGTCAGCCTGAGCTACAGATGCCCATGCCGATTCTTCGAAAGCCATGTTGCCAGAGCCA
ACGTCAAGCATCTCAAAATTCTCAACACCCCAAACTGTGCCCTTCAGATTGTAGCCCGGCTGAAGAACA
ACAACAGACAAGTGTGCATTGACCCGAAGCTAAAGTGGATTCAGGAGTACCTGGAGAAAGCCTTAAACA
AGTAATCTAGAGGGCCCTATTCTATAGTGTCACCTAAATGCTAGAGCTCGCTGATCAGCCTCGACTGTG
CCTTCTAGTTGCCAGCCATCTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAGTAGGTGTCATTCTATTCTG
GGGGGTGGGGTGGGGCAGGACAGCAAGGGGGAGGATTGGGAAGACAATAGCAGGCATGCTGGGGATGCG
GTGGGCTCTATGGCTTCTGAGGCGGAAAGAACCAGGGCCGCGGTGGCCATCATGACCAAAATCCCTTAA
CGTGAGTTTTCGTTCCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTGGTTTGTTTGCCGGAT
CAAGAGCTACCAACTCTTTTTCCGAAGGTAACTGGCTTCAGCAGAGCGCAGATACCAAATACTGTTCTT
CTAGTGTAGCCGTAGTTAGGCCACCACTTCAAGAACTCTGTAGCACCGCCTACATACCTCGCTCTGCTA
ATCCTGTTACCAGTGGCTGCTGCCAGTGGCGATAAGTCGTGTCTTACCGGGTTGGACTCAAGACGATAG
TTACCGGATAAGGCGCAGCGGTCGGGCTGAACGGGGGGTTCGTGCACACAGCCCAGCTTGGAGCGAACG
ACCTACACCGAACTGAGATACCTACAGCGTGAGCTATGAGAAAGCGCCACGCTTCCCGAAGGGAGAAAG
GCGGACAGGTATCCGGTAAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGATTTTTGTGATGCTCG
TCAGGGGGGCGGAGCCTATGGAAAAACGCCAGCAACGCGGCCTTTTTACGGTTCCTGGCCTTTTGCTGG
CCTTTTGCTCACATGAATTCAGAAGAACTCGTCAAGAAGGCGATAGAAGGCGATGCGCTGCGAATCGGG
AGCGGCGATACCGTAAAGCACGAGGAAGCGGTCAGCCCATTCGCCGCCAAGCTCTTCAGCAAATCACGG
GTAGCCAACGCTATGTCCTGATAGCGGTCCGCCACACCCAGCCGGCCACAGTCGATGAATCCAGAAAAG
CGGCCATTTTCCACCATGATATTCGGCAAGCAGGCATCGCCATGGGTCACGACGAGATCCTCGCCGTCG
GGCATGCTCGCCTTGAGCCTGGCGAACAGTTCGGCTGGCGCGAGCCCCTGATGCTCTTCGTCCAGATCA
TCCTGATCGACAAGACCGGCTTCCATCCGAGTACGTGCTCGCTCGATGCGATGTTTCGCTTGGTGGTCG
AATGGGCAGGTAGCCGGATCAAGCGTATGCAGCCGCCGCATTGCATCAGCCATGATGGATACTTTCTCG
GCAGGAGCAAGGTGAGATGACAGGAGATCCTGCCCCGGCACTTCGCCCAATAGCAGCCAGTCCCTTCCC
GCTTCAGTGACAACGTCGAGCACAGCTGCGCAAGGAACGCCCGTCGTGGCCAGCCACGATAGCCGCGCT
GCCTCGTCTTGCAGTTCATTCAGGGCACCGGACAGGTCGGTCTTGACAAAAAGAACCGGGCGCCCCTGC
GCTGACAGCCGGAACACGGCGGCATCAGAGCAGCCGATTGTCTGTTGTGCCCAGTCATAGCCGAATAGC
CTCTCCACCCAAGCGGCCGGAGAACCTGCGTGCAATCCATCTTGTTCAATCATGCGAAACGATCCTCAT
CCTGTCTCTTGATC

The complete disclosure of all patents, patent applications, and publications, and electronically available material cited herein are incorporated by reference. The foregoing detailed description and examples have been given for clarity of understanding only. No unnecessary limitations are to be understood there from. The invention is not limited to the exact details shown and described, for variations obvious to one skilled in the art will be included within the invention defined by the claims.

What is claimed is:

1. A method for treating an anal or sphincter wound of a subject, comprising administering a therapeutically effective amount of a stromal cell-derived factor-1 (SDF-1) protein or protein variant, or an SDF-1 or SDF-1 variant expression vector in or proximate to the anal wound.

2. The method of claim 1, wherein an SDF-1 expression vector is administered to the subject.

3. The method of claim 2, wherein the SDF-1 expression vector is a plasmid vector.

4. The method of claim 2, wherein the SDF-1 expression vector comprises SEQ ID NO: 6.

5. The method of claim 1, wherein an SDF-1 protein comprising SEQ ID NO: 1 is administered to the subject.

6. The method of claim 1, wherein the SDF-1 protein or SDF-1 expression vector is injected into the wound or an area proximate to the wound.

7. The method of claim 1, wherein the method further comprises administering mesenchymal stem cells in or proximate to the anal wound.

8. The method of claim 1, wherein the SDF-1 protein or SDF-1 expression vector is administered as a topical formulation.

9. The method of claim 8, wherein the topical formulation comprises a hydrogel scaffold.

10. The method of claim 1, wherein the SDF-1 protein or SDF-1 expression vector is administered at least one week after the anal injury occurred.

11. The method of claim 1, wherein the SDF-1 protein or SDF-1 expression vector is administered at least 30 days after the anal injury occurred.

12. The method of claim 1, wherein an anal wound is treated.

13. The method of claim 12, wherein the anal wound is a chronic anal wound.

14. The method of claim 12, wherein the anal wound is an anal sphincter wound.

15. The method of claim 12, wherein the anal wound is a muscle defect.

16. The method of claim 1, wherein a sphincter wound is treated.

17. A topical formulation for treating an anal or sphincter wound, comprising a topical pharmaceutical carrier and an SDF-1 protein or protein variant, or an SDF-1 or SDF-1 variant expression vector.

18. The topical formulation of claim 17, wherein the SDF-1 expression vector is a plasmid vector.

19. The topical formulation of claim 18, wherein the SDF-1 expression vector comprises SEQ ID NO: 6.

20. The topical formulation of claim 17, wherein the topical formulation further comprises mesenchymal stem cells.

21. The topical formulation of claim 17, wherein the topical pharmaceutical carrier comprises a hydrogel scaffold.

22. The topical formulation of claim 17, wherein the formulation comprises an SDF-1 protein comprising SEQ ID NO: 1.
